(19) United States
(12) Patent Application Publication
(10) Pub. No.: US 2007/0009922 A1
(43) Pub. Date: Jan. 11, 2007

(54) Hot start polymerase reaction using a thermolabile blocker

(75) Inventor: Michael Borns, Escondido, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/324,887

(22) Filed: Jan. 4, 2006

Related U.S. Application Data
(60) Provisional application No. 60/641,197, filed on Jan. 4, 2005.

(51) Int. Cl. C12Q 1/68; C07H 21/04; C12P 21/06; C12P 19/34; C12N 9/22

(52) U.S. Cl. 435/6; 435/91.2; 435/199; 435/69.1; 435/320.1; 435/325; 536/23.2

(57) ABSTRACT

The invention relates to compositions, methods, and kits for hot start polynucleotide synthesis, including extension of primed polynucleotide templates and polymerase chain reaction (PCR). Hot start is provided by a thermally inactivated blocking polymerase protein that binds primed polynucleotide templates and prevents their access to a thermostable nucleic acid polymerase. High temperatures employed in the synthesis reaction cause the blocking polymerase to denature, thereby permitting the action of a thermostable processive polymerase. Compositions of the invention include a specific blocking polymerase protein which is a mutant of the Klenow fragment of E. coli DNA polymerase. The mutant is essentially devoid of polymerase activity, processivity, and 3′ to 5′ exonuclease activity. Use of the thermally inactivated blocking polymerase together with a thermostable polymerase reduces non-specific priming and accumulation of unwanted amplification products, increasing the specificity and sensitivity of the synthesis reaction.

This application claims the benefit of U.S. Provisional Application No. 60/641,197, filed on Jan. 4, 2005, which is incorporated herein by reference in its entirety.

### TECHNICAL FIELD

The invention relates to compositions and methods for the amplification or extension of nucleic acid sequences.

### BACKGROUND

In the polymerase chain reaction (PCR), amplification of non-target oligonucleotides due to side-reactions such as mispriming on non-target nucleic acids or the primers themselves, is a significant problem. This is especially true in diagnostic PCR applications, where amplification is carried out in the presence of background nucleic acids and the target may be present low levels, even down to a single copy (Chou et al., Nucleic Acid Res., 20:1717-1723 (1992)).

While thermostable polymerases such as Taq exhibit their highest activity at temperatures in the range of 70° C., they also possess significant activity at lower temperatures in the range of 20 to 37° C. Thus, during set up for PCR at ambient temperatures, extension at non-specific sequences can occur due to the formation of only a few base pairs at the 3′-end of a primer. The resulting products can be competitive or inhibitory. Primer dimers are formed by the action of DNA polymerase on primers paired with each other, independent of the target template. The probability of primer-primer interactions increases with the number of primer pairs in the reaction, and is increased with multiplex PCR. Nonspecific priming on the template DNA can produce incorrect bands of various size. The resulting nonspecific extension products can compete with the desired target DNA or may confuse the interpretation of results.

The “hot start” PCR method is designed to minimize side reactions, thereby improving yield and specificity. Hot start PCR may be accomplished by various physical, chemical, or biochemical methods. Physical hot start methods rely on separating the DNA polymerase or one or more reaction components from the sample DNA until high temperature is reached. Physical hot start can be achieved using a wax barrier, as disclosed in U.S. Pat. Nos. 5,599,660 and 5,411,876. See also Hebert et al., Mol. Cell Probes 7:249-252 (1993); Horton et al., Biotechniques 16:42-43 (1994). Another hot start method involves a chemically inactivated DNA polymerase, such as AMPLITAQ GOLD™ by PE Applied Biosystems. The enzyme is provided in an inactivated form which can be activated by heating. A further hot start method is to combine the DNA polymerase with an antibody against the polymerase. One such method employs a monoclonal, inactivating antibody raised against Taq DNA polymerase. See Scalice et al., J. Immun. Methods 172: 147-163 (1994); Sharkey et al., Biotechnology 12:506-509 (1994); and Kellogg et al., Biotechniques 16: 1134-1137 (1994). The antibody inhibits the polymerase activity at ambient temperature but is inactivated at higher temperatures by heat denaturation. Another method for hot start PCR involves special primers with secondary structures that prevent them from annealing until denatured at cycling temperatures. See Ailenberg et al., Biotechniques 29: 1018-1020 and 1022-1024 (2000).

Present methods of performing hot start PCR are tedious, expensive, or have other shortcomings. In wax methods, for example, the wax hardens after the amplification and tends to plug pipet tips used to remove the sample. Wax methods also suffer from cross-contamination problems and interference with the PCR reaction. With chemically inactivated polymerases, the reactivation conditions (95° C. for 10 minutes) are harsh enough to depurinate the template DNA. These enzymes are most efficient when amplifying a small target DNA of approximately 200 base pairs; they cannot be used with targets longer than a few kilobases. Antibodies which inhibit thermostable polymerases are expensive and must be used in large excess, which can interfere with PCR. Specially designed primers are usually longer than standard primers and must be carefully designed.

Accordingly, there is a need for novel and improved methods of hot start PCR.

### SUMMARY OF THE INVENTION

The invention is related to novel compositions and methods for nucleic acid amplification and extension.

The invention provides compositions useful for hot start amplification of a target nucleic acid sequence by PCR. In particular, the invention provides a thermolabile blocker, which is a blocking polymerase. The blocking polymerase binds to a primed polynucleotide template and, below its inactivation temperature, blocks elongation of the primer by the thermostable polymerase. The blocking polymerase has an inactivation temperature below the priming temperature of the thermostable polymerase. Preferably, the blocking polymerase becomes inactivated above 37° C., 45° C., 50° C., 60° C., 70° C., or 80° C. Preferably, the blocking polymerase becomes inactivated below the temperature at which the thermostable polymerase carries out elongation of a primed polynucleotide template. More preferably, the blocking polymerase becomes inactivated below 90° C., 85° C., 80° C., 75° C., 70° C., 65° C., 60° C., 55° C., or 50° C.

In a preferred embodiment, the blocking polymerase is a functionally deficient nucleic acid polymerase. More preferably the blocking polymerase is a mutant DNA polymerase which is deficient in polymerase activity and 3′ to 5′ exonuclease activity. In some embodiments, the blocking polymerase has low processivity or is non-processive. In some embodiments the blocking polymerase is a naturally ocurring protein in isolated form. In a preferred embodiment, the blocking polymerase is a mutant derived from the Klenow fragment of E. coli Pol I. In a more preferred embodiment the blocking polymerase is a polypeptide comprising the amino acid sequence shown in SEQ ID NO:1.

The invention also provides a mutant DNA polymerase comprising the amino acid sequence shown in SEQ ID NO:1. The invention further provides a nucleic acid molecule comprising a nucleotide sequence that encodes the amino acid sequence shown in SEQ ID NO:1, for example, a nucleic acid molecule comprising the nucleotide sequence depicted in SEQ ID NO:2. The invention moreover comprises vectors and host cells comprising a nucleic acid molecule having a nucleotide sequence that encodes the amino acid sequence shown in SEQ ID NO:1.

Another aspect of the invention is a composition comprising a thermostable processive nucleic acid polymerase and a blocking polymerase. The nucleic acid polymerase can be a DNA polymerase, such as Taq DNA polymerase, Pfu DNA polymerase, or a mixture of Taq and Pfu DNA polymerases. The blocking polymerase binds to a primed polynucleotide template and, below its inactivation temperature, blocks elongation of the primer by the thermostable polymerase. The blocking polymerase has an inactivation temperature below the priming temperature of the thermostable polymerase. Preferably, the blocking polymerase becomes inactivated above 37° C., 45° C., 50° C., 60° C., 70° C., or 80° C. Preferably, the blocking polymerase becomes inactivated below the temperature at which the thermostable polymerase carries out elongation of a primed polynucleotide template. More preferably, the blocking polymerase becomes inactivated below 90° C., 85° C., 80° C., 75° C., 70° C., 65° C., 60° C., 55° C., or 50° C. In a preferred embodiment, the blocking polymerase is a functionally deficient nucleic acid polymerase. More preferably the blocking polymerase is a mutant DNA polymerase which is deficient in polymerase activity and 3′ to 5′ exonuclease activity. In some embodiments, the blocking polymerase has low processivity or is non-processive. In some embodiments the blocking polymerase is a naturally ocurring protein in isolated form. In a preferred embodiment, the blocking polymerase is a mutant derived from the Klenow fragment of E. coli Pol I. In a more preferred embodiment the blocking polymerase is a polypeptide comprising the amino acid sequence shown in SEQ ID NO:1. Preferably, the priming temperature of the processive polymerase is higher than the inactivation temperature of the blocking polymerase. In some embodiments, the concentration of the blocking polymerase in the composition is greater than the concentration of the processive polymerase. In other embodiments, the concentration of the blocking polymerase is less than or equal to the concentration of the processive polymerase, and the blocking polymerase has a higher affinity than the processive polymerase for binding to the primed polynucleotide template at temperatures below the inactivation temperature of the blocking polymerase. In some embodiments of this composition, the blocking polymerase is a nucleic acid polymerase and the composition includes an antibody that partially or completely inhibits the polymerase activity of the blocking polymerase. The composition may include different amounts of the antibody, such as an amount of antibody sufficient to bind 10%, 20%, 30%, 40%, 50%, or more of the blocking polymerase protein. The polymerase activity of the blocking polymerase can be inhibited by 5%, 10%, 15%, 20%, 30%, 40%, 50% or more by the antibody in the composition.

In additional aspects of the invention, the compositions are part of a kit for amplifying a target nucleic acid sequence in a sample. The kit provides a blocking polymerase of a thermostable polymerase. The blocking polymerase binds to a primed polynucleotide template and, below its inactivation temperature, blocks elongation of the primer by the thermostable polymerase. The blocking polymerase has an inactivation temperature below the priming temperature of the thermostable polymerase. Preferably, the blocking polymerase becomes inactivated above 37° C., 45° C., 50° C., 60° C., 70° C., or 80° C. Preferably, the blocking polymerase becomes inactivated below the temperature at which the thermostable polymerase carries out elongation of a primed polynucleotide template. More preferably, the blocking polymerase becomes inactivated below 90° C., 85° C., 80° C., 75° C., 70° C., 65° C., 60° C., 55° C., or 50° C. In a preferred embodiment, the blocking polymerase is a functionally deficient nucleic acid polymerase. More preferably the blocking polymerase is a mutant DNA polymerase which is deficient in polymerase activity and 3′ to 5′ exonuclease activity. In some embodiments, the blocking polymerase has low processivity or is non-processive. In some embodiments the blocking polymerase is a naturally ocurring protein in isolated form. In a preferred embodiment, the blocking polymerase is a mutant derived from the Klenow fragment of E. coli Pol I. In a more preferred embodiment the blocking polymerase is a polypeptide comprising the amino acid sequence shown in SEQ ID NO:1. In some versions of the kit, the blocking polymerase is a nucleic acid polymerase and the kit includes an antibody that partially or completely inhibits the polymerase activity of the blocking polymerase. For example, the polymerase activity of the blocking polymerase can be inhibited by 5%, 10%, 15%, 20%, 30%, 40%, 50% or more by the antibody. Furthermore, the kit may include different amounts of the antibody, such as an amount of antibody sufficient to bind 10%, 20%, 30%, 40%, 50%, or more of the blocking polymerase protein.

The kit optionally includes a thermostable processive polymerase. The nucleic acid polymerase can be, for example, a DNA polymerase, such as Taq DNA polymerase, Pfu DNA polymerase, or a mixture of Taq and Pfu DNA polymerases.

The kit may further include a suitable buffer, a primer, a mixture of deoxyribonucleotides, and packaging material therefor.

Another embodiment of the invention is a method of primer extension. The method comprises comprising extending an oligonucleotide primer which is annealed to a nucleic acid template using a mixture of a thermostable processive polymerase and a blocking polymerase. The blocking polymerase is added prior to initiating the extension reaction, while the reaction mixture is below the inactivation temperature of the blocking polymerase. The extension reaction is performed at a temperature above the inactivation temperature of the blocking polymerase. In some embodiments the thermostable processive polymerase and the blocking polymerase are added to the reaction mixture at about the same time. In other embodiments, the blocking polymerase is added to the reaction mixture prior to adding the thermostable processive polymerase. The blocking polymerase binds to a primed polynucleotide template and, below its inactivation temperature, blocks elongation of the primer by the thermostable polymerase.

The invention also provides a method for carrying out hot start PCR. The method comprises performing a PCR reaction in the presence of a thermostable processive polymerase and a blocking polymerase. The blocking polymerase is added prior to initiating the first extension reaction, while the reaction mixture is below the inactivation temperature of the blocking polymerase. The first extension reaction is performed at a temperature above the inactivation temperature of the blocking polymerase. In some embodiments the thermostable processive polymerase and the blocking polymerase are added to the reaction mixture at about the same time. In other embodiments, the blocking polymerase is added to the reaction mixture prior to adding the thermostable processive polymerase. The blocking polymerase binds to a primed polynucleotide template and, below its inactivation temperature, blocks elongation of the primer by the thermostable polymerase.

In the hot start PCR methods of the invention, the thermostable processive polymerase can be a DNA polymerase, such as Taq DNA polymerase, Pfu DNA polymerase, or a mixture of Taq and Pfu DNA polymerases. The blocking polymerase has an inactivation temperature below the priming temperature of the thermostable polymerase. Preferably, the blocking polymerase becomes inactivated above 37° C., 45° C., 50° C., 60° C., 70° C., or 80° C. Preferably, the blocking polymerase becomes inactivated below the temperature at which the thermostable polymerase carries out elongation of a primed polynucleotide template. More preferably, the blocking polymerase becomes inactivated below 90° C., 85° C., 80° C., 75° C., 70° C., 65° C., 60° C., 55° C., or 50° C. In a preferred embodiment, the blocking polymerase is a functionally deficient nucleic acid polymerase. More preferably the blocking polymerase is a mutant DNA polymerase which is deficient in polymerase activity and 3′ to 5′ exonuclease activity. In some embodiments, the blocking polymerase has low processivity or is non-processive. In some embodiments the blocking polymerase is a naturally ocurring protein in isolated form. In a preferred embodiment, the blocking polymerase is a mutant derived from the Klenow fragment of E. coli Pol I. In a more preferred embodiment the blocking polymerase is a polypeptide comprising the amino acid sequence shown in SEQ ID NO:1.

### BRIEF DESCRIPTION OF DRAWINGS

FIG. 1 shows the amino acid sequence (SEQ ID NO:1) of a mutant of the Klenow fragment of E. coli DNA polymerase I. Mutated residues are indicated by underlining.

FIG. 2 shows a nucleotide sequence (SEQ ID NO:2) encoding the mutant of the Klenow fragment of E. coli DNA polymerase I whose amino acid sequence is shown in FIG. 1. Mutated residues are indicated by underlining.

FIGS. 3A and 3B show the DNA polymerase activity of D705P Exo(−) Klenow DNA polymerase relative to Exo(−) Klenow DNA polymerase (FIG. 3A) and PfuTurbo® (FIG. 3B) in the primed M13 DNA polymerase activity assay. All reactions were incubated for 3 hours at 25° C. Activity is represented as incorporation of radioactively labeled nucleotide. FIG. 3A: Lane 1, 1.25U Exo(−) Klenow; Lane 2, 90 ng (weight equivalent of 7.5U of native Exo(−) Klenow) D705P Exo(−) Klenow. FIG. 3B: Lane 1, 1.25U PfuTurbo®; Lane 2, 15 ng (weight equivalent of 1.25U of native Exo(−) Klenow) D705P Exo(−) Klenow.

FIG. 4 shows measurement of primed substrate blocking by D705P Exo(−) Klenow DNA Polymerase in the primed M13 DNA polymerase activity assay. Lanes 1-6 contain 1.25U PfuTurbo® DNA polymerase. Lane 1: 0 ng D705P Exo(−) Klenow. Lane 2: 7.5 ng D705P Exo(−) Klenow. Lane 3: 15 ng D705P Exo(−) Klenow. Lane 4: 30 ng D705P Exo(−) Klenow. Lane 5: 60 ng D705P Exo(−) Klenow. Lane 6: 90 ng D705P Exo(−) Klenow. All reactions were incubated at 25° C. for 3 hours. Activity is represented as incorporation of radioactively labeled nucleotide.

FIG. 5 shows PCR hot start evaluation of D705P Exo(−) Klenow DNA Polymerase in the 115 bp HIV gag PCR hot start assay with PfuTurbo® DNA polymerase. Lanes 1-5 contain 2.5U PfuTurbo®. Lane1: 2.5U PfuTurbo®+0 ng D705P Exo(−) Klenow. Lane 2: 2.5U PfuTurbo®+7.5 ng D705P Exo(−) Klenow. Lane3: 2.5U PfuTurbo®+15 ng D705P Exo(−) Klenow. Lane 4: 2.5U PfuTurbo®+30 ng D705P Exo(−) Klenow. Lane 5: 2.5U PfuTurbo®+60 ng D705P Exo(−) Klenow. Lane 6: 2.5U PfuTurbo® hot start with hot start antibody (positive hot start control). Lane M: Phi-X174/Hinf I Marker (Stratagene Catalog No. 201102), containing 21 fragments ranging in size from 24 bp to 726 bp. All reactions were incubated at 25° C. for 15 minutes before thermal cycling.

FIG. 6 shows a PCR comparison of D705P Exo(−) Klenow DNA Polymerase hot start and the hot start antibody technology in the 115 bp HIV gag PCR hot start assay with Taq 2000® DNA polymerase. Lanes 1-3 contain 2.5U Taq 2000®. Lane1: 2.5U Taq 2000®+0 ng D705P Exo(−) Klenow. Lane 2: 2.5U Taq 2000®+3.5 ng D705P Exo(−) Klenow. Lane 3: 2.5U Taq 2000®+7.5 ng D705P Exo(−) Klenow. Lane 4: 2.5U Taq 2000® hot start with hot start antibody. Lane M: Phi-X174/Hinf I Marker (Stratagene Catalog No. 201102), containing 21 fragments ranging in size from 24 bp to 726 bp. All reactions were incubated at 25° C. for 15 minutes before thermal cycling.

FIG. 7 shows a PCR comparison of D705P Exo(−) Klenow DNA Polymerase hot start and the hot start antibody technology in the 115 bp HIV gag PCR hot start assay with Herculase® DNA polymerase. Lanes 1 and 2 contain 2.5U Herculase®. Lane1: 2.5U Herculase®+0 ng D705P Exo(−) Klenow. Lane2: 2.5U Herculase®+60 ng D705P Exo(−) Klenow. Lane 3: 2.5U Herculase® hot start with hot start antibody. Lane M: Phi-X174/Hinf I Marker (Stratagene Catalog No. 201102), containing 21 fragments ranging in size from 24 bp to 726 bp. All reactions were incubated at 25° C. for 15 minutes before thermal cycling.

### DETAILED DESCRIPTION OF THE INVENTION

The invention provides a novel method to achieve hot start polynucleotide extension or amplification, including hot start PCR, by using a thermally sensitive blocking polymerase protein that binds to a primed polynucleotide substrate, thereby blocking extension or amplification by a processive polymerase. The blocking polymerase protein prevents access by the processive polymerase to the primed polynucleotide substrate until the blocking polymerase protein is thermally inactivated. Following thermal inactivation of the blocking polymerase, the processive polymerase has access to the primed substrate and is then free to extend or amplify the primed polynucleotide substrate.

The thermally sensitive blocking polymerase protein can be any protein that interacts with a primed polynucleotide substrate so as to substantially inhibit the action of the processive polymerase used for extension or amplification. One example of such a blocking polymerase protein is a mutant DNA polymerase which retains the capability of binding a primed polynucleotide template but is essentially non-processive, has essentially lost its polymerase activity, and is inactivated at the high temperatures commonly used for amplification in PCR. One partictular mutant which has been developed by the inventor, the D705P exo(−) mutant of the Klenow fragment of E. coli Pol I, effectively blocks primed substrate from thermostable processive DNA polymerases and has been demonstrated to function as effectively as existing hot start antibody technology, which uses an antibody to a thermostable polymerase to reduce its activity below the thermal denaturation temperature for the antibody. Because the D705P exo(−) Klenow mutant DNA polymerase binds the primed substrate it can be used as a universal hot start technology for any processive polymerase that has higher thermostability.

General Definitions

As used herein, a “polynucleotide” refers to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The term “polynucleotide” includes, without limitation, single- and double-stranded polynucleotide. The term “polynucleotide” as it is employed herein embraces chemically, enzymatically or metabolically modified forms of polynucleotide. “Polynucleotide” also embraces a short polynucleotide, often referred to as an oligonucleotide (e.g., a primer or a probe). A polynucleotide has a “5′-terminus” and a “3′-terminus” because polynucleotide phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus. As used herein, a polynucleotide sequence, even if internal to a larger polynucleotide (e.g., a sequence region within a polynucleotide), also can be said to have 5′- and 3′-ends.

As used herein, the term “oligonucleotide” refers to a short polynucleotide, typically less than or equal to 150 nucleotides long (e.g., between 5 and 150, preferably between 10 to 100, more preferably between 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

As used herein, the term “complementary” refers to the concept of sequence complementarity between regions of two polynucleotide strands or between two regions of the same polynucleotide strand. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” refers to a first polynucleotide that is 100% or “fully” complementary to a second polynucleotide and thus forms a base pair at every nucleotide position. “Complementary” also refers to a first polynucleotide that is not 100% complementary (e.g., 90%, or 80% or 70% complementary) contains mismatched nucleotides at one or more nucleotide positions. In one embodiment, two complementary polynucleotides are capable of hybridizing to each other under high stringency hybridization conditions. For example, for membrane hybridization (e.g., Northern hybridization), high stringency hybridization conditions are defined as incubation with a radiolabeled probe in 5×SSC, 5× Denhardt's solution, 1% SDS at 65° C. Stringent washes for membrane hybridization are performed as follows: the membrane is washed at room temperature in 2×SSC/0.1% SDS and at 65° C. in 0.2×SSC/0.1% SDS, 10 minutes per wash, and exposed to film.

As used herein, the term “hybridization” or “binding” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

As used herein, when one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition.

As used herein, “Tₘ” and “melting temperature” are interchangeable terms which are the temperature at which 50% of a population of double-stranded polynucleotide molecules becomes dissociated into single strands. The equation for calculating the Tₘ of polynucleotides is well known in the art. For example, the Tₘ may be calculated by the following equation: Tₘ=69.3+0.41×(G+C)%−650/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically.

As used herein, the term “template” refers to that strand of a nucleic acid molecule from which a complementary nucleic acid strand is synthesized by a nucleic acid polymerase. As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, “synthesis” refers to any in vitro method for making a new strand of polynucleotide or elongating existing polynucleotide (i.e., DNA or RNA) in a template dependent manner. Synthesis, according to the invention, includes amplification, which increases the number of copies of a polynucleotide template sequence with the use of a polymerase. Polynucleotide synthesis (e.g., amplification) results in the incorporation of nucleotides into a polynucleotide (i.e., a primer), thereby forming a new polynucleotide molecule complementary to the polynucleotide template. The formed polynucleotide molecule and its template can be used as templates to synthesize additional polynucleotide molecules.

As used herein, “nucleic acid polymerase” refers to an enzyme that catalyzes the polymerization of nucleotides. Generally, the enzyme will initiate synthesis at the 3′-end of the primer annealed to a nucleic acid template sequence, and will proceed toward the 5′ end of the template strand. “DNA polymerase” catalyzes the polymerization of deoxynucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), 9°Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzyme can be determined by means well known in the art. One unit of DNA polymerase activity, according to the subject invention, is defined as the amount of enzyme which catalyzes the incorporation of 10 nmoles of total dNTPs into polymeric form in 30 minutes at optimal temperature (e.g., 72° C. for Pfu DNA polymerase).

As used herein, “5′ to 3′ exonuclease activity” or “5′→3′ exonuclease activity” refers to that activity of a template-specific nucleic acid polymerase e.g. a 5′→3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, a “primer” refers to a type of oligonucleotide having or containing the length limits of an “oligonucleotide” as defined above, and having or containing a sequence complementary to a target polynucleotide, which hybridizes to the target polynucleotide through base pairing so to initiate an elongation (extension) reaction to incorporate a nucleotide into the oligonucleotide primer. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 100 nucleotides in length, preferably between about 17 and 50 nucleotides in length, and most preferably between about 17 and 45 nucleotides in length. An “amplification primer” is a primer for amplification of a target sequence by primer extension. As no special sequences or structures are required to drive the amplification reaction, amplification primers for PCR may consist only of target binding sequences. A “primer region” is a region on a “oligonucleotide probe” or a “bridging oligonucleotide probe” which hybridizes to the target nucleic acid through base pairing so to initiate an elongation reaction to incorporate a nucleotide into the oligonucleotide primer.

“Primer extension reaction” or “synthesizing a primer extension” means a reaction between a target-primer hybrid and a nucleotide which results in the addition of the nucleotide to a 3′-end of the primer such that the incorporated nucleotide is complementary to the corresponding nucleotide of the target polynucleotide. Primer extension reagents typically include (i) a polymerase enzyme; (ii) a buffer; and (iii) one or more extendible nucleotides.

As used herein, “polymerase chain reaction” or “PCR” refers to an in vitro method for amplifying a specific polynucleotide template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and polynucleotide template. One PCR reaction may consist of 5 to 100 “cycles” of denaturation and synthesis of a polynucleotide molecule. The PCR process is described in U.S. Pat. Nos. 4,683,195 and 4,683,202, the disclosures of which are incorporated herein by reference.

As used herein, “target nucleic acid” refers to a nucleic acid containing an amplified region. The “amplified region,” as used herein, is a region of a nucleic acid that is to be either synthesized or amplified by polymerase chain reaction (PCR). For example, an amplified region of a nucleic acid template resides between two sequences to which two PCR primers are complementary to.

As used herein, an “amplified product” refers to the double stranded polynucleotide population at the end of a PCR amplification reaction. The amplified product contains the original polynucleotide template and polynucleotide synthesized by DNA polymerase using the polynucleotide template during the PCR reaction.

The term “wild-type” refers to a gene or gene product which has the characteristics of that gene or gene product when isolated from a naturally occurring source. In contrast, the term “modified” or “mutant” refers to a gene or gene product which displays altered characteristics when compared to the wild-type gene or gene product. For example, a mutant DNA polymerase in the present invention is a DNA polymerase which exhibits a reduced uracil detection activity.

As used herein, the term “sample” refers to a biological material which is isolated from its natural environment and containing a polynucleotide. A “sample” according to the invention may consist of purified or isolated polynucleotide, or it may comprise a biological sample such as a tissue sample, a biological fluid sample, or a cell sample comprising a polynucleotide. A biological fluid includes blood, plasma, sputum, urine, cerebrospinal fluid, lavages, and leukophoresis samples. A sample of the present invention may be a plant, animal, bacterial or viral material containing a target polynucleotide. Useful samples of the present invention may be obtained from different sources, including, for example, but not limited to, from different individuals, different developmental stages of the same or different individuals, different disease individuals, normal individuals, different disease stages of the same or different individuals, individuals subjected to different disease treatment, individuals subjected to different environmental factors, individuals with predisposition to a pathology, individuals with exposure to an infectious disease (e.g., HIV). Useful samples may also be obtained from in vitro cultured tissues, cells, or other polynucleotide containing sources. The cultured samples may be taken from sources including, but are not limited to, cultures (e.g., tissue or cells) cultured in different media and conditions (e.g., pH, pressure, or temperature), cultures (e.g., tissue or cells) cultured for different period of length, cultures (e.g., tissue or cells) treated with different factors or reagents (e.g., a drug candidate, or a modulator), or cultures of different types of tissue or cells.

As used herein, a polynucleotide “isolated” from a sample is a naturally occurring polynucleotide sequence within that sample which has been removed from its normal cellular (e.g., chromosomal) environment. Thus, an “isolated” polynucleotide may be in a cell-free solution or placed in a different cellular environment.

As used herein, the term “amount” refers to an amount of a target polynucleotide in a sample, e.g., measured in μg, μmol or copy number. The abundance of a polynucleotide in the present invention is measured by the fluorescence intensity emitted by such polynucleotide, and compared with the fluorescence intensity emitted by a reference polynucleotide, i.e., a polynucleotide with a known amount.

Polymerases

The nucleic acid polymerases used in the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the methods of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,18S; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22(15):3259-3260 (1994)). For amplification of long nucleic acid molecules (e.g., nucleic acid molecules longer than about 3-5 Kb in length), at least two DNApolymerases are typically used, one substantially lacking 3′ exonuclease activity and the other having 3′ exonuclease activity. See U.S. Pat. No. 5,436,149; U.S. Pat. No. 5,512,462; Fames, W. M., Gene 112:29-35 (1992); and copending U.S. patent application Ser. No. 09/741,664, filed Dec. 21, 2000, the disclosures of which are incorporated herein in their entireties. Examples of DNA polymerases substantially lacking in 3′ exonuclease activity include, but are not limited to, Taq, Tne (exo −), Tma (exo −), Pfu (exo −), Pwo (exo −) and Tth DNA polymerases, and mutants, variants and derivatives thereof.

As used herein, “thermostable” refers to an enzyme which is stable and active at temperatures as great as preferably between about 90-100° C. and more preferably between about 70-98° C. to heat as compared, for example, to a non-thermostable form of an enzyme with a similar activity. For example, a thermostable nucleic acid polymerase derived from thermophilic organisms such as P. furiosus, M. jannaschii, A. fulgidus or P. horikoshii are more stable and active at elevated temperatures as compared to a nucleic acid polymerase from E. coli. A representative thermostable nucleic acid polymerase isolated from P. furiosus (Pfu) is described in Lundberg et al., 1991, Gene, 108:1-6. Additional representative temperature stable polymerases include, e.g., polymerases extracted from the thermophilic bacteria Thermus flavus, Thermus ruber, Thermus thermophilus, Bacillus stearothermophilus (which has a somewhat lower temperature optimum than the others listed), Thermus lacteus, Thermus rubens, Thermotoga maritima, or from thermophilic archaea Thermococcus litoralis, and Methanothermus fervidus.

For PCR amplifications, the enzymes used in the invention are preferably thermostable. As used herein, “thermostable” refers to an enzyme which is stable to heat, is heat resistant, and functions at high temperatures, e.g., 50 to 90° C. The thermostable enzyme according to the present invention must satisfy a single criterion to be effective for the amplification reaction, i.e., the enzyme must not become irreversibly denatured (inactivated) when subjected to the elevated temperatures for the time necessary to effect denaturation of double-stranded polynucleotides. By “irreversible denaturation” as used in this connection, is meant a process bringing a permanent and complete loss of enzymatic activity. The heating conditions necessary for denaturation will depend, e.g., on the buffer salt concentration and the length and nucleotide composition of the polynucleotides being denatured, but typically range from 85° C., for shorter polynucleotides, to 105° C. for a time depending mainly on the temperature and the polynucleotide length, typically from 0.25 minutes for shorter polynucleotides, to 4.0 minutes for longer pieces of DNA. Higher temperatures may be tolerated as the buffer salt concentration and/or GC composition of the polynucleotide is increased. Preferably, the enzyme will not become irreversibly denatured at 90 to 100° C. An enzyme that does not become irreversibly denatured, according to the invention, retains at least 10%, or at least 25%, or at least 50% or more function or activity during the amplification reaction.

As used herein, “archaeal” DNA polymerase refers to a DNA polymerase that belong to either the Family B/pol I-type group (e.g., Pfu, KOD, Pfx, Vent, Deep Vent, Tgo, Pwo) or the pol II group (e.g., Pyrococcus firiosus DP1/DP2 2-subunit DNA polymerase). “Archaeal” DNA polymerase refers to a thermostable DNA polymerases useful in PCR and includes, but is not limited to, DNA polymerases isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus. It is estimated that suitable archaea exhibit maximal growth temperatures of >80-85° C. or optimal growth temperatures of >70-80° C. Appropriate PCR enzymes from the archaeal pol I DNA polymerase group are commercially available, including Pfu (Stratagene), KOD (Toyobo), Pfx (Life Technologies, Inc.), Vent (New England BioLabs), Deep Vent (New England BioLabs), Tgo (Roche), and Pwo (Roche). Additional archaea related to those listed above are described in Archaea: A Laboratory Manual (Robb, F. T. and Place, A. R., eds.), Cold Spring Harbor Laboratory Press, Cold Spring Harbor, N.Y., 1995.

As used herein, useful “Taq” DNA polymerase includes wild type Taq DNA polymerase and mutant forms of Taq DNA polymerase with reduced fidelity (e.g., Patel et al., 2001, J. Biol. Chem. 276: 5044, hereby incorporated by reference).

Some naturally occurring thermostable DNA polymerases possess enzymatically active 3′ to 5′ exonuclease domains, providing a natural proofreading capability and, thus, exhibiting higher fidelity than Taq DNA polymerase. However, these DNA polymerases also show slower DNA extension rates and an overall lower processivity when compared to Taq DNA polymerase.

Multiple enzyme assemblages can also be used in PCR, for example, combining Taq polymerase and a proofreading enzyme, such as Pfu polymerase or Vent DNA polymerase. Such multiple-enzyme mixtures exhibit higher PCR efficiency and reduced error rates when compared to Taq polymerase alone (Barnes, PNAS USA 91:2216-2220 (1994)).

Useful variants of Taq polymerase have been developed through deletion/truncation techniques. The Stoffel fragment, for example, is a 544 amino acid C-terminal truncation of Taq DNA polymerase, possessing an enzymatically active 5′ to 3′ polymerase domain but lacking 3′ to 5′ exonuclease and 5′ to 3′ exonuclease activity. Other commercially available thermostable polymerase deletions include Vent (exo−) and Deep Vent (exo−) (New England Biolabs, Beverly, Mass.).

As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

As used herein, “proofreading” activity refers to 3′ to 5′ exonuclease activity of a DNA polymerase. A “non-proofreading” enyzme refers to a DNA polymerase that is “3′ to 5′ exonuclease deficient” or “3′ to 5′exo-”. As used herein, “3′ to 5′exonuclease deficient” or “3′ to 5′exo-” refers to an enzyme that substantially lacks the ability to remove incorporated nucleotides from the 3′ end of a DNA polymer. DNA polymerase exonuclease activities, such as the 3′ to 5′ exonuclease activity exemplified by members of the Family B polymerases, can be lost through mutation, yielding an exonuclease-deficient polymerase. As used herein, a DNA polymerase that is deficient in 3′ to 5′ exonuclease activity substantially lacks 3′ to 5′ exonuclease activity. “Substantially lacks” encompasses a lack of activity of, for example, 0.03%, 0.05%, 0.1%, 1%, 5%, 10%, 20%, 50%, or even complete lack of the exonuclease activity relative to the parental enzyme. Methods used to generate and characterize 3′ to 5′ exonuclease DNA polymerases as well as mutations that reduce or eliminate 3′ to 5′ exonuclease activity are disclosed in the pending U.S. patent application Ser. No. 09/698,341 (Sorge et al; filed Oct. 27, 2000). Additional mutations that reduce or eliminate 3′ to 5′ exonuclease activity are known in the art and contemplated herein.

There are a variety of commercially available Pol I DNA polymerases, some of which have been modified to reduce or abolish 5′ to 3′ exonuclease activity. Methods used to eliminate 5′ to 3′ exonuclease activity of pol I DNA polymerases include: mutagenesis (as described in Xu et al., 1997, J. Mol. Biol., 268:284 and Kim et al., 1997, Mol. Cells, 7:468); N-truncation by proteolytic digestion (as described in Klenow et al., 1971, Eur. J. Biochem., 22: 371); or N-truncation by cloning and expressing as C-terminal fragments (as described in Lawyer et al., 1993, PCR Methods Appl., 2:275).

The invention also contemplates DNA polymerases in combination with accessory factors, for example as described in U.S. Pat. No. 6,333,158, and WO 01/09347 A2, hereby incorporated by reference in its entirety.

Polymerase Chain Reaction

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

Nucleic acid amplification results in the incorporation of nucleotides into a nucleic acid (e.g., DNA) molecule or primer thereby forming a new nucleic acid molecule complementary to the nucleic acid template. The formed nucleic acid molecule and its template can be used as templates to synthesize additional nucleic acid molecules. As used herein, one amplification reaction may consist of many rounds of nucleic acid synthesis. Amplification reactions include, for example, polymerase chain reactions (PCR; Mullis and Faloona, 1987, Methods Enzymol., 155:335). One PCR reaction may consist of 5 to 100 “cycles” of denaturation and synthesis of a nucleic acid molecule. PCR amplifications with an exo-DNA polymerase inherently will result in generating mutated amplified product.

The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and nucleic acid template. The PCR reaction comprises providing a set of oligonucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of oligonucleotide primers” or “a set of PCR primers” can comprise two, three, four or more primers.

A PCR primer can be a single stranded DNA or RNA molecule that can hybridize to a nucleic acid template and prime enzymatic synthesis of a second nucleic acid strand. A PCR primer useful according to the invention is between 10 to 100 nucleotides in length, preferably 17-50 nucleotides in length and more preferably 17-45 nucleotides in length.

Probes and primers are typically prepared by biological or chemical synthesis, although they can also be prepared by biological purification or degradation, e.g., endonuclease digestion.

For short sequences such as probes and primers used in the present invention, chemical synthesis is frequently more economical as compared to biological synthesis. For longer sequences standard replication methods employed in molecular biology can be used such as the use of M13 for single stranded DNA as described by Messing, 1983, Methods Enzymol. 101: 20-78. Chemical methods of polynucleotide or oligonucleotide synthesis include phosphotriester and phosphodiester methods (Narang, et al., Meth. Enzymol. (1979) 68:90) and synthesis on a support (Beaucage, et al., Tetrahedron Letters. (1981) 22:1859-1862) as well as phosphoramidate technique, Caruthers, M. H., et al., Methods in Enzymology (1988)154:287-314 (1988), and others described in “Synthesis and Applications of DNA and RNA,” S. A. Narang, editor, Academic Press, New York, 1987, and the references contained therein.

Use of a labeled probe generally in conjunction with the amplification of a target polynucleotide, for example, by PCR, e.g., is described in many references, such as Innis et al., editors, PCR Protocols (Academic Press, New York, 1989); Sambrook et al., Molecular Cloning, Second Edition (Cold Spring Harbor Laboratory, New York, 1989), all of which are hereby incorporated herein by reference. In some embodiments, the binding site of the probe is located between the PCR primers used to amplify the target polynucleotide. In other embodiments, the oligonucleotide probe complex acts as a primer. In another embodiment, the oligonucleotide probe complex binds to a target nucleic acid present in a primer incorporated into the amplicon. Preferably, PCR is carried out using Taq DNA polymerase, e.g., Amplitaq (Perkin-Elmer, Norwalk, Conn.), or an equivalent thermostable DNA polymerase, and the annealing temperature of the PCR is about 5° C. to 10° C. below the melting temperature of the oligonucleotide probes employed.

A PCR reaction buffer may contain any known chemicals used in a buffer for PCR reaction. Preferably, the buffer contains a buffering composition selected from Tris or Tricine. More preferably, the buffering composition has a pH range of from 7.5 to 9.5. Preferably, the universal PCR reaction buffer contains Mg²⁺ (e.g., MgCl₂ or MgSO₄) in the range of 1-10 mM. The buffer according to the invention may also contain K⁺ (e.g., KCl) in the range of from 0 to 20 mM. In some embodiments, the buffer contains components which enhances PCR yield (e.g., (NH₄)₂SO₄ in the range of from 0 to 20 mM). In other embodiments, the buffer contains one or more non-ionic detergents (e.g., Trition X-100, Tween 20, or NP40, in the range of from 0 to 1%). The buffer may also contain BSA in the range of from 1-100 μg/ml. In a preferred embodiment of the invention, the universal PCR reaction buffer contains 10 mM KCl, 10 mM (NH₄)₂SO₄, 20 mM Tris-Cl (pH 8.8), 2 mM MgSO₄, 0.1% Triton X-100, 100 μg/ml BSA. In another preferred embodiment, the buffer contains 10 mM KCl, 10 mM (NH₄)₂SO₄, 20 mM Tris-Cl (pH 9.2), 2 mM MgSO₄, 0.1% Triton X-100, 100 μg/ml BSA.

As used herein, the term “repeating one or more additional subsequent PCR amplification reactions” refers to the subsequent performance of one or more additional PCR amplification reactions comprising incubating a nucleic acid template, at least two PCR primers, an error-prone DNA polymerase under conditions which permit amplification of the nucleic acid template. A subsequent PCR reaction comprises said incubating step using the PCR amplified product of a preceding PCR amplification as template. The amplified product of a preceding PCR amplification reaction may be purified before being used as template for a subsequent PCR reaction by means known in the art, e.g., phenol extraction/ethanol precipitation or column purification. The template for a subsequent PCR amplification reaction may be a portion of or the total amplified product of a preceding PCR amplification. For each subsequent PCR amplification, fresh reagents (e.g., reaction buffer, dNTP, DNA polymerase, primers) are added to the reaction mixture. If a portion of the amplified product of a preceding PCR amplification is used, the volume of a subsequent PCR reaction may be the same as the preceding PCR reaction. If the total amplified product of a preceding PCR reaction is used as template, a subsequent PCR reaction will have larger volume than the preceding PCR reaction.

The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and polynucleotide template. PCR requires two primers that hybridize with the double-stranded target polynucleotide sequence to be amplified. In PCR, this double-stranded target sequence is denatured and one primer is annealed to each strand of the denatured target. The primers anneal to the target polynucleotide at sites removed from one another and in orientations such that the extension product of one primer, when separated from its complement, can hybridize to the other primer. Once a given primer hybridizes to the target sequence, the primer is extended by the action of a DNA polymerase. The extension product is then denatured from the target sequence, and the process is repeated.

In successive cycles of this process, the extension products produced in earlier cycles serve as templates for DNA synthesis. Beginning in the second cycle, the product of amplification begins to accumulate at a logarithmic rate. The amplification product is a discrete double-stranded DNA molecule comprising: a first strand which contains the sequence of the first primer, eventually followed by the sequence complementary to the second primer, and a second strand which is complementary to the first strand.

Due to the enormous amplification possible with the PCR process, small levels of DNA carryover from samples with high DNA levels, positive control templates or from previous amplifications can result in PCR product, even in the absence of purposefully added template DNA. If possible, all reaction mixes are set up in an area separate from PCR product analysis and sample preparation. The use of dedicated or disposable vessels, solutions, and pipettes (preferably positive displacement pipettes) for RNA/DNA preparation, reaction mixing, and sample analysis will minimize cross contamination. See also Higuchi and Kwok, 1989, Nature, 339:237-238 and Kwok, and Orrego, in: Innis et al. eds., 1990, PCR Protocols: A Guide to Methods and Applications, Academic Press, Inc., San Diego, Calif., which are incorporated herein by reference.

PCR enhancing factors may also be used to improve efficiency of the amplification. As used herein, a “PCR enhancing factor” or a “Polymerase Enhancing Factor” (PEF) refers to a complex or protein possessing polynucleotide polymerase enhancing activity (Hogrefe et al., 1997, Strategies 10::93-96; and U.S. Pat. No. 6,183,997, both of which are hereby incorporated by references). For Pfu DNA polymerase, PEF comprises either P45 in native form (as a complex of P50 and P45) or as a recombinant protein. In the native complex of Pfu P50 and P45, only P45 exhibits PCR enhancing activity. The P50 protein is similar in structure to a bacterial flavoprotein. The P45 protein is similar in structure to dCTP deaminase and dUTPase, but it functions only as a dUTPase converting dUTP to dUMP and pyrophosphate. PEF, according to the present invention, can also be selected from the group consisting of: an isolated or purified naturally occurring polymerase enhancing protein obtained from an archeabacteria source (e.g., Pyrococcus furiosus); a wholly or partially synthetic protein having the same amino acid sequence as Pfu P45, or analogs thereof possessing polymerase enhancing activity; polymerase-enhancing mixtures of one or more of said naturally occurring or wholly or partially synthetic proteins; polymerase-enhancing protein complexes of one or more of said naturally occurring or wholly or partially synthetic proteins; or polymerase-enhancing partially purified cell extracts containing one or more of said naturally occurring proteins (U.S. Pat. No. 6,183,997, supra). The PCR enhancing activity of PEF is defined by means well known in the art. The unit definition for PEF is based on the dUTPase activity of PEF (P45), which is determined by monitoring the production of pyrophosphate (PPi) from dUTP. For example, PEF is incubated with dUTP (10 mM dUTP in 1× cloned Pfu PCR buffer) during which time PEF hydrolyzes dUTP to dUMP and PPi. The amount of PPi formed is quantitated using a coupled enzymatic assay system that is commercially available from Sigma (#P7275). One unit of activity is functionally defined as 4.0 nmole of PPi formed per hour (at 85° C.).

Other PCR additives may also affect the accuracy and specificity of PCR reactions. EDTA less than 0.5 mM may be present in the amplification reaction mix. Detergents such as Tween-20™ and Nonidet™ P-40 are present in the enzyme dilution buffers. A final concentration of non-ionic detergent approximately 0.1% or less is appropriate, however, 0.01-0.05% is preferred and will not interfere with polymerase activity. Similarly, glycerol is often present in enzyme preparations and is generally diluted to a concentration of 1-20% in the reaction mix. Glycerol (5-10%), formamide (1-5%) or DMSO (2-10%) can be added in PCR for template DNA with high GC content or long length (e.g., >1 kb). These additives change the Tm (melting temperature) of primer-template hybridization reaction and the thermostability of polymerase enzyme. BSA (up to 0.8 μg/μl) can improve efficiency of PCR reaction. Betaine (0.5-2M) is also useful for PCR over high GC content and long fragments of DNA. Tetramethylammonium chloride (TMAC, >50 mM), Tetraethylammonium chloride (TEAC), and Trimethlamine N-oxide (TMANO) may also be used. Test PCR reactions may be performed to determine optimum concentration of each additive mentioned above.

The invention provides for additives including, but not limited to antibodies (for hot start PCR) and ssb (single strand DNA binding protein; higher specificity). The invention also contemplates mutant archael DNA polymerases in combination with accessory factors, for example as described in U.S. Pat. No. 6,333,158, and WO 01/09347 A2, hereby incorporated by reference in its entirety.

Various specific PCR amplification applications are available in the art (for reviews, see for example, Erlich, 1999, Rev Immunogenet., 1:127-34; Prediger 2001, Methods Mol. Biol. 160:49-63; Jurecic et al., 2000, Curr. Opin. Microbiol. 3:316-21; Triglia, 2000, Methods Mol. Biol. 130:79-83; MaClelland et al., 1994, PCR Methods Appl. 4:S66-81; Abramson and Myers, 1993, Current Opinion in Biotechnology 4:41-47; each of which is incorporated herein by reference).

The subject invention can be used in PCR applications including, but not limited to: (i) Hot-start PCR which reduces non-specific amplification. (ii) Nested PCR which synthesizes more reliable product using an outer set of primers and an inner set of primers. (iii) Inverse PCR for amplification of regions flanking a known sequence. In this method, DNA is digested, the desired fragment is circularized by ligation, then PCR using primer complementary to the known sequence extending outwards. (iv) AP-PCR (arbitrarily primed)/RAPD (randomly amplified polymorphic DNA). These methods create genomic fingerprints from species with little-known target sequences by amplifying using arbitrary oligonucleotides. (v) RT-PCR which uses RNA-directed DNA polymerase (e.g., reverse transcriptase) to synthesize cDNAs which is then used for PCR. This method is extremely sensitive for detecting the expression of a specific sequence in a tissue or cell. It may also be used to quantify mRNA transcripts. (vi) RACE (rapid amplification of cDNA ends). This is used where information about DNA/protein sequence is limited. The method amplifies 3′ or 5′ ends of cDNAs generating fragments of cDNA with only one specific primer each (plus one adaptor primer). Overlapping RACE products can then be combined to produce full length cDNA. (vii) DD-PCR (differential display PCR) which is used to identify differentially expressed genes in different tissues. The first step in DD-PCR involves RT-PCR, then amplification is performed using short, intentionally nonspecific primers. (viii) Multiplex-PCR in which two or more unique targets of DNA sequences in the same specimen are amplified simultaneously. One DNA sequence can be used as a control to verify the quality of PCR. (ix) Q/C-PCR (Quantitative comparative) which uses an internal control DNA sequence (but of a different size) which competes with the target DNA (competitive PCR) for the same set of primers. (x) Recusive PCR which is used to synthesize genes. Oligonucleotides used in this method are complementary to stretches of a gene (>80 bases), alternately to the sense and to the antisense strands with ends overlapping (˜20 bases). (xi) Asymmetric PCR. (xii) In Situ PCR. (xiii) Site-directed PCR Mutagenesis.

This invention is not limited to any particular amplification system. As other systems are developed, those systems may benefit by practice of this invention.

Temperature stable polymerases are preferred in a thermocycling process wherein double stranded nucleic acids are denatured by exposure to a high temperature (about 95° C.) during the PCR cycle.

One of average skill in the art may also employ other PCR parameters to increase the fidelity of the synthesis/amplification reaction. It has been reported that PCR fidelity may be affected by factors such as changes in dNTP concentration, units of enzyme used per reaction, pH, and the ratio of Mg²⁺ to dNTPs present in the reaction (Mattila et al., 1991, supra).

Mg²⁺ concentration affects the annealing of the oligonucleotide primers to the template DNA by stabilizing the primer-template interaction, it also stabilizes the replication complex of polymerase with template-primer. It can therefore also increase non-specific annealing and produce undesirable PCR products (gives multiple bands in gel). When non-specific amplification occurs, Mg²⁺ may need to be lowered or EDTA can be added to chelate Mg²⁺ to increase the accuracy and specificity of the amplification.

Other divalent cations such as Mn²⁺, or Co²⁺ can also affect DNA polymerization. Suitable cations for each DNA polymerase are known in the art (e.g., in DNA Replication 2^{nd} edition, supra). Divalent cation is supplied in the form of a salt such MgCl₂, Mg(OAc)₂, MgSO₄, MnCl₂, Mn(OAc)₂, or MnSO₄. Usable cation concentrations in a Tris-HCl buffer are for MnCl₂ from 0.5 to 7 mM, preferably, between 0.5 and 2 mM, and for MgCl₂ from 0.5 to 10 mM. Usable cation concentrations in a Bicine/KOAc buffer are from 1 to 20 mM for Mn(OAc)₂, preferably between 2 and 5 mM.

Monovalent cation required by DNA polymerase may be supplied by the potassium, sodium, ammonium, or lithium salts of either chloride or acetate. For KCl, the concentration is between 1 and 200 mM, preferably the concentration is between 40 and 100 mM, although the optimum concentration may vary depending on the polymerase used in the reaction.

Deoxyribonucleoside triphosphates (dNTPs) are added as solutions of the salts of dATP, dCTP, dGTP, dUTP, and dTTP, such as disodium or lithium salts. In the present method, a final concentration in the range of 1 μM to 2 mM each is suitable, and 100-600 μM is preferable, although the optimal concentration of the nucleotides may vary in the PCR reaction depending on the total dNTP and divalent metal ion concentration, and on the buffer, salts, particular primers, and template. For longer products, i.e., greater than 1500 bp, 500 μM of each dNTP may be preferred when using a Tris-HCl buffer.

dNTPs chelate divalent cations; therefore the amount of divalent cations used may need to be changed according to the dNTP concentration in the reaction. Excessive amount of dNTPs (e.g., larger than 1.5 mM) can increase the error rate and possibly inhibit DNA polymerases. Lowering the dNTP (e.g., to 10-50 μM) may reduce the error rate. PCR reactions for amplifying larger size templates may need more dNTPs.

The pH of the buffering component in standard PCR reaction buffers is from 8.3-8.8. However, other pH ranges may be used as appropriate. For example, a different pH range may be required by the particular goals of the amplification process or by an enzyme or any other component of the reaction mixture.

PCR is a very powerful tool for DNA amplification and therefore very little template DNA is needed. However, in some embodiments, to reduce the likelihood of error, a higher DNA concentration may be used. However, too many templates may increase the amount of contaminants and reduce efficiency.

Usually, up to 3 μM of primers may be used, but high primer to template ratio can result in non-specific amplification and primer-dimer formation. Therefore it is usually necessary to check primer sequences to avoid primer-dimer formation.

Denaturation time may be increased if the template GC content is high. Higher annealing temperature may be needed for primers with high GC content or longer primers. Gradient PCR is a useful way of determining the annealing temperature. Extension time should be extended for larger PCR product amplifications. However, the extension time may need to be reduced whenever possible to limit damage to the polymerase. The number of cycles can be increased if the template number is very low, and decreased if template number is high.

Thermally Sensitive Blocking Polymerase Proteins

A thermally sensitive blocking polymerase protein of the invention can be any protein that interacts with a primed polynucleotide substrate so as to inhibit the action of the processive polymerase used for extension or amplification. The blocking polymerase protein can bind the 3′ end of a primed template and block a thermally stable processive polymerase until the blocking polymerase protein is thermally inactivated during an extension or amplification reaction. The blocking polymerase protein does not dislodge the primer from the polynucleotide substrate. In a preferred embodiment, the blocking polymerase protein is a functionally deficient nucleic acid polymerase which retains the capability of binding a primed polynucleotide template, but has limited, or completely absent, polymerase activity. Also preferred is a nucleic acid polymerase with limited, or completely absent, processivity. In some preferred embodiments the polymerase has limited, or completely absent, 3′ to 5′ exonuclease activity. In other preferred embodiments, the polymerase has limited, or completely absent, 5′ to 3′ exonuclease activity. More preferred are embodiments in which the blocking polymerase is a mutant DNA polymerase which is essentially non-processive, has essentially lost its polymerase activity, and becomes inactivated as a blocking polymerase at the high temperatures commonly used for amplification in PCR. Further examples of thermally sensitive blocking polymerase proteins include the DNA end binding protein human KU (see, e.g., Lee et al., Mol. Cells 13:159-166 (2002)), E. coli exonuclease III which has been mutated to knock out 3′ to 5′ exonuclease activity (see, e.g., Taft-Benz et al., Nucleic Acids Res. 26:4005-4011 (1998)) while retaining DNA binding activity, human WRN protein (see, e.g., J. Biol. Chem. 279:53465-74 (2004)) which has been mutated to knock out 3′ to 5′ exonuclease activity while retaining DNA binding activity, and Drosophila melanogaster endo-exo DmGEN protein (see, e.g., Nucleic Acids Res. 32:6251-9 (2004)) which has been mutated to knock out 3′ to 5′ exonuclease activity while retaining DNA binding activity.

The catalytic palm domain of the Klenow fragment of E. coli Pol I contains a highly conserved 13 amino acid motif known as Motif A. All of the amino acids of Motif A can be mutated with some retention of activity, with the exception of the catalytically essential aspartic acid residue at position 705. Shinkai et al., J. Biol. Chem. 276:18336-42 (2001). Therefore, mutation of Asp⁷⁰⁵ of the Klenow fragment can be used to generate thermally sensitive blocking polymerase proteins according to the invention.

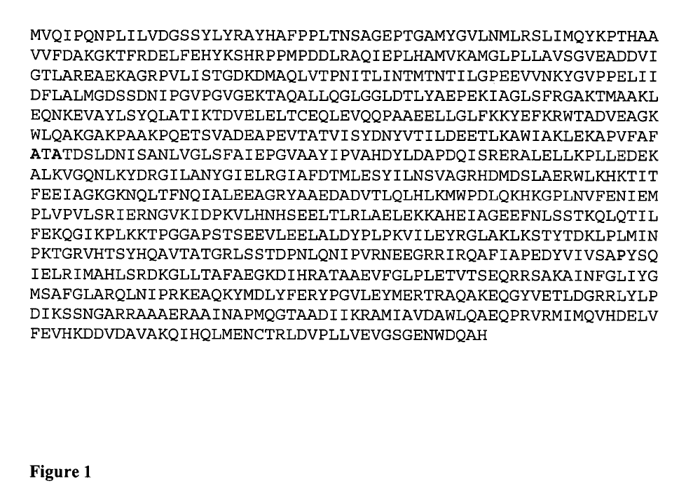

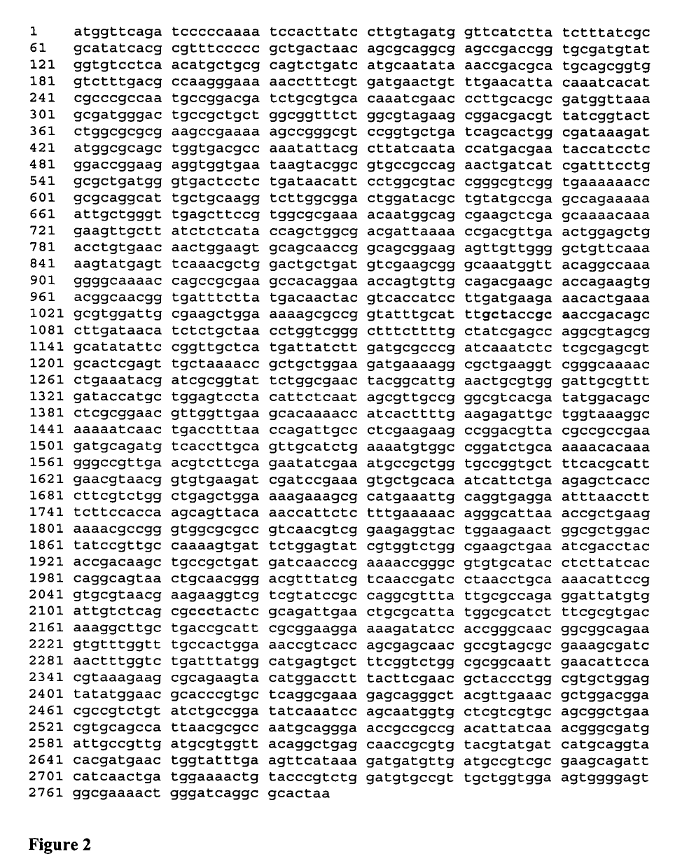

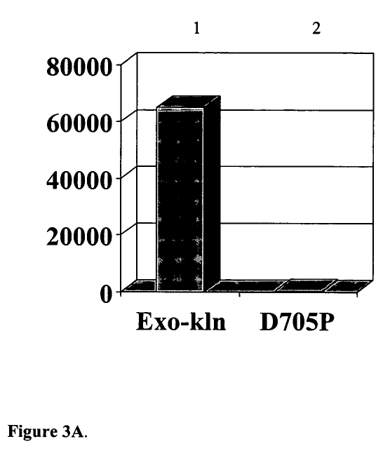

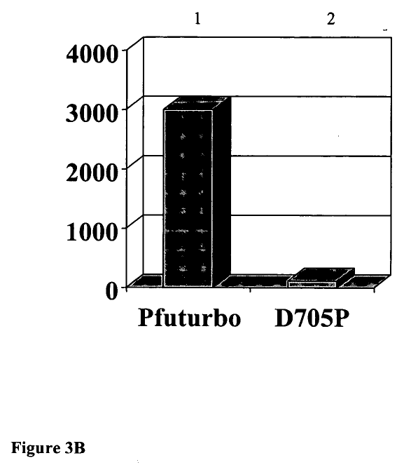

One partictular preferred blocking polymerase is a mutant DNA polymerase which is the D705P exo(−) mutant of the Klenow fragment of E. coli Pol I, in which the aspartic acid residue at position 705 has been replaced with proline, resulting in essentially complete loss of polymerase activity (see FIGS. 3A, 3B and Example 1). This mutant is also devoid of 3′ to 5′ exonuclease activity, by virtue of two additional mutations, D355A and E357A. The amino acid sequence of this mutant is shown in FIG. 1 and is represented in the sequence listing as SEQ ID NO:1. A polynucleotide seuqence that encodes the amino acid sequence of this mutant is shown in FIG. 2 and as SEQ ID NO:2. The polynucleotide sequence depicted in SEQ ID NO:2, or a similar sequence employing the degeneracy of the genetic code, can be inserted into an expression vector and used to express the D705P (exo−) Klenow mutant. This mutant binds to primed polynucleotide templates and effectively blocks synthesis (extension or amplification) by a thermostable processive DNA polymerase. It has been demonstrated to function in hot start PCR as effectively as an antibody to a thermostable polymerase. See Examples below. Because the D705P exo(−) Klenow mutant DNA polymerase binds the primed substrate it can be used as a universal hot start technology for any processive polymerase that has higher thermostability.

A blocking polymerase protein according to the invention is thermosensitive in the sense that its function as a blocking polymerase is reduced at above ambient temperatures typically encountered during PCR or reactions involving thermostable polymerases. Blocking polymerase activity is largely intact at ambient temperatures, or temperatures that might be encountered during set up for PCR, such as from about 0° C. to about 37° C. The blocking polymerase protein is more thermosenstive than the polymerase used for synthesis, although the blocking polymerase need not be any more thermosensitive than an average protein. The thermosensitivity of a given blocking polymerase protein can be described in terms of its “inactivation temperature”, defined herein as the temperature at which blocking ability (i.e., inhibition of the activity of a processive nucleic acid polymerase) is reduced to 50% of the optimum blocking activity. Optimum blocking activity would typically be observed somewhere in the range of 0° C. to 37° C. Preferably, the inactivation temperature of the blocking polymerase protein is below the temperature at which nucleic acid synthesis (elongation or amplification) is carried out. The polymerase used for synthesis should be sufficiently stable (i.e., thermostable) at or above the inactivation temperature of the blocking polymerase so as to allow the elongation or amplification reaction to be reasonably completed prior to inactivation of the polymerase. The inactivation temperature of a blocking polymerase according to the invention can be at least about 37, 45, 50, 60, 70, or 80 C, and is preferably in the range of 40° C. to 50° C., 50° to 60° C., or 60° to 70° C. The blocking polymerase protein becomes inactivated below the temperature at which the thermostable polymerase carries out elongation of a primed polynucleotide template. Preferably, the blocking polymerase has an inactivation temperature below 90° C., 85° C., 80° C., 75° C., 70° C., 65° C., 60° C., 55° C., or 50° C. By way of example, the inactivation temperature of the D705P (exo−) Klenow mutant is approximately 50° C.

The thermally sensitive blocking polymerase of the invention can be a polymerase with reduced or deficient polymerase activity. Generation of such a polymerase can be accomplished by mutagenesis of any amino acid, mutagenesis of any combination or group of amino acids, or truncation at any amino acid, such that polymerase activity is reduced. However, substrate binding activity should not be reduced to the point of eliminating the ability of the blocking polymerase to bind a primed polynucleotide template. Polymerase activity can also be reduced or eliminated by any chemical modification that would reduce polymerase activity, but would not affect binding activity to the point of eliminating the ability of the polymerase to bind a primed polynucleotide template. The polymerase activity could also be reduced or eliminated by an antibody or fragment of an antibody (e.g., Fab) that would reduce polymerase activity but not substrate binding activity to the point of eliminating the ability of the polymerase to bind substrate. The polymerase could be a native polymerase that has low or deficient polymerase activity.

The thermally sensitive blocking polymerase protein of the present invention has the ability to bind single stranded and double stranded DNA, DNA primed templates, and any polynucleotide primed polynucleotide template.

As used herein, “mutant” polymerase refers to a DNA polymerase comprising one or more mutations that modulate one or more activities of the DNA polymerase including, but limited to, DNA polymerization activity, base analog detection activities, reverse transcriptase activity, processivity, salt resistance, DNA binding, strand displacement activity, nucleotide binding and recognition, 3′ to 5′ or 5′ to 3′ exonuclease activities, proofreading, fidelity, or decreases DNA polymerization at room temperature. A “mutant” polymerase as defined herein, includes a polymerase comprising one or more amino acid substitutions, one or more amino acid insertions, a truncation, or an internal deletion. A “mutant” polymerase as defined herein includes non-chimeric and chimeric polymerases.

Any chemical modification method or mutation known in the art can be used to reduce polymerase activity of a nucleic acid polymerase in order to produce a blocking polymerase suitable for hot start reactions. For example, methods used to generate Pfu DNA polymerases with reduced DNA polymerization activity are disclosed in pending U.S. patent application Ser. No. 10/035,091 (Hogrefe, et al.; filed: Dec. 21, 2001); pending U.S. patent application Ser. No. 10/079,241 (Hogrefe, et al.; filed Feb. 20, 2002); pending U.S. patent application Ser. No. 10/208,508 (Hogrefe et al.; filed Jul. 30, 2002); pending U.S. patent application Ser. No. 10/227,110 (Hogrefe et al.; filed Aug. 23, 2002); and pending U.S. patent application Ser. No. 10/324,846 (Borns et al.; filed Dec. 20, 2002), the contents of which are hereby incorporated in their entirety. As used herein, a polymerase with a “reduced” or “deficient” “polymerase activity” or “polimerization activity” is a polymerase mutant comprising a DNA polymerization activity which is lower than that of the wild-type enzyme, e.g., comprising less than 50%, 20%, 10%, 8%, 6%, 4%, 2%, or less than 1% of the polymerization activity of that of the wild-type enzyme.

The thermally sensitive blocking polymerase of the invention can be a polymerase with reduced or deficient 3′ to 5′ exonuclease activity. The 3′ to 5′ exonuclease activity of a polymerase can be reduced by mutagenesis of any amino acid, mutagenesis of any combination or group of amino acids, or by truncation at any amino acid that would reduce 3′ to 5′ exonuclease activity but not affect binding activity to the point of eliminating the ability of the polymerase to bind a primed polynucleotide template. The 3′ to 5′ exonuclease activity could also be reduced or eliminated by any chemical modification that would reduce 3′ to 5′ exonuclease activity but not affect binding activity to the point of eliminating the ability of the polymerase to bind a primed polynucleotide template. The 3′ to 5′ exonuclease activity could also be reduced or eliminated by an antibody or fragment of an antibody (e.g., Fab) that would affect 3′ to 5′ exonuclease activity but not binding activity to the point of eliminating the ability of the polymerase to bind a primed polynucleotide substrate. The polymerase could be a native polymerase that has low or deficient 3′ to 5′ exonuclease activity.

In preferred embodiments the blocking polymerase is a mutant polymerase with reduced processivity or which is essentially non-processive. As used herein, “processivity” refers to the ability of a nucleic acid modifying enzyme, for example a polymerase, to remain attached to the template or substrate and perform multiple modification reactions, including polymerization of nucleotides. “Processivity” also refers to the ability of a nucleic acid modifying enzyme, for example a DNA polymerase, to perform a sequence of polymerization steps without intervening dissociation of the enzyme from the growing DNA chains. “Processivity” can depend on the nature of the polymerase, the sequence of a DNA template, and reaction conditions, for example, salt concentration, temperature or the presence of specific proteins.

There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to 3′ exonuclease activity, but in some cases 3′ to 5′ exonuclease activity is located in the same polypeptide. There are no commercial sources of eubacterial pol II and pol III DNA polymerases. There are a variety of commercially available Pol I DNA polymerases, some of which have been modified to reduce or abolish 5′ to 3′ exonuclease activity.

Polymerase mutagenesis has been used to develop new and useful nucleic acid polymerase variants. For example, naturally occurring DNA polymerases strongly discriminate against the incorporation of nucleotide analogues. This property contributes to the fidelity of DNA replication and repair. However, the incorporation of nucleotide analogues is useful for many DNA synthesis applications, especially DNA sequencing. Hence, a DNA polymerase that lacks associated exonucleolytic activity, either 5′-nuclease activity or 3′ to 5′ exonuclease activity, is preferred for DNA sequencing. In order to generate thermostable DNA polymerases with reduced nucleotide discrimination, site-directed mutagenesis studies were initiated and resulted in the identification of mutant forms of a number of thermostable DNA polymerases with the requisite activities suitable for DNA sequencing (U.S. Pat. No. 5,466,591, incorporated herein by reference).

Yet another approach to modifying the property of a DNA polymerase is to generate DNA polymerase fusions in which one or more protein domains having the requisite activity are combined with a DNA polymerase. DNA polymerase has been fused in frame to the helix-hairpin-helix DNA binding motifs from DNA topoisomerase V and shown to increase processivity, salt resistance and thermostability of the chimeric DNA polymerase as described in Pavlov et al., 2002, Proc. Natl. Acad. Sci USA, 99:13510-13515. Fusion of the thioredoxin binding domain to T7 DNA polymerase enhances the processivity of the DNA polymerase fusion in the presence of thioredoxin as described in WO 97/29209, U.S. Pat. No. 5,972,603 and Bedford et al. Proc. Natl. Acad. Sci. USA 94: 479-484 (1997). Fusion of the archaeal PCNA binding domain to Taq DNA polymerase results in a DNA polymerase fusion that in the presence of PCNA has enhanced processivity and produces higher yields of PCR amplified DNA (Motz, M., et al., J. Biol. Chem. 2002 May 3; 277 (18); 16179-88). Also, fusion of the sequence non-specific DNA binding protein Sso7d or Sac7d from Sulfolobus sulfataricus to a DNA polymerase, such as Pfu or Taq DNA polymerase, was shown to greatly increase the processivity of these DNA polymerases as disclosed in WO 01/92501 A1 which is hereby incorporated by reference in its entirety. Domain substitution of all or a portion of a DNA polymerase with the corresponding domain of a different DNA polymerase has also been described (U.S. 2002/0119461).

Direct comparison of DNA polymerases from diverse organisms indicates that the domain structure of these enzymes is highly conserved and in many instances, it is possible to assign a particular function to a well-defined domain of the enzyme. For example, the six most conserved C-terminal regions, spanning approximately 340 amino acids, are located in the same linear arrangement and contain highly conserved motifs that form the metal and dNTP binding sites and the cleft for holding the DNA template and are therefore essential for the polymerization function. In another example, the three amino acid regions containing the critical residues in the E. coli DNA polymerase I involved in metal binding, single-stranded DNA binding, and catalysis of the 3′-5′ exonuclease reaction are located in the amino-terminal half and in the same linear arrangement in several prokaryotic and eukaryotic DNA polymerases. The location of these conserved regions provides a useful model to direct genetic modifications for preparing mutant DNA polymerase with modified activities while conserving desired functions e.g. template recognition.

For example, a mutant DNA polymerase can be generated by genetic modification (e.g., by modifying the DNA sequence of a wild-type DNA polymerase). A number of methods are known in the art that permit the random as well as targeted mutation of DNA sequences (see for example, Ausubel et. al. Short Protocols in Molecular Biology (1995) 3^{rd} Ed. John Wiley & Sons, Inc.). In addition, there are a number of commercially available kits for site-directed mutagenesis, including both conventional and PCR-based methods. Examples include the EXSITE™ PCR-Based Site-directed Mutagenesis Kit available from Stratagene (Catalog No. 200502) and the QUIKCHANGE™ Site-directed mutagenesis Kit from Stratagene (Catalog No. 200518), and the CHAMELEON® double-stranded Site-directed mutagenesis kit, also from Stratagene (Catalog No. 200509).

Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Methods of random mutagenesis, which will result in a panel of mutants bearing one or more randomly situated mutations, exist in the art. Such a panel of mutants may then be screened for desired activity such as those exhibiting properties including but not limited to reduced DNA polymerization activity, 3′-5′ exonuclease deficiency, or processivity relative to the wild-type polymerase (e.g., by measuring the incorporation of 10 nmoles of dNTPs into polymeric form in 30 minutes in the presence of 200 μM dUTP and at the optimal temperature for a given DNA polymerase). An example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

Genes for desired mutant DNA polymerases generated by mutagenesis may be sequenced to identify the sites and number of mutations. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

Typically, the 5′ to 3′ exonuclease activity, 3′ to 5′ exonuclease activity, discriminatory activity, and fidelity can be affected by substitution of amino acids typically which have different properties. For example, an acidic amino acid such as Asp may be changed to a basic, neutral or polar but uncharged amino acid such as Lys, Arg, His (basic); Ala, Val, Leu, Ile, Pro, Met, Phe, Trp (neutral); or Gly, Ser, Thr, Cys, Tyr, Asn or Gln (polar but uncharged). Glu may be changed to Asp, Ala, Val Leu, Ile, Pro, Met, Phe, Trp, Gly, Ser, Thr, Cys, Tyr, Asn or Gln.

Methods of measuring the efficiency of a DNA polymerase are described in PCR Primer: A Laboratory Manual, 1995, CSHL Press, Cha and Thilly, pp. 37-51. Methods of measuring template length amplification capability are described in Proc Natl. Acad. Sci USA, 2002, 99:596-601 and J. Biotechnol., 2001, 88:141-149. Methods of measuring specificity of a DNA polymerase are described in J. Biochem. (Tokyo), 1999, 126:762-8. Methods of measuring thermostability of a DNA polymerase are described in FEMS Microbiol. Lett, 2002, 217:89-94. Methods of measuring nucleotide binding and recognition are described in J. Mol. Biol., 2002, 322:719-729 and Nucleic Acids Res., 2002, 30:605-13.

Use of a Temperature Sensitive Blocking Polymerase in Hot Start Reactions

To achieve hot start polynucleotide synthesis, the thermally sensitive blocking polymerase protein is added to a polynucleotide synthesis reaction with a thermally stable processive polymerase or a polymerase that is more active than the blocking polymerase above the inactivation temperature of the blocking polymerase. The blocking polymerase achieves hot start by binding to nonspecifically primed templates at room temperature or suboptimal priming temperatures and making them unavailable to the processive polymerase for extension. This effectively stops the processive polymerase from producing non-specific extension or amplification products, which is the goal of “hot start” synthetic techniques. When the temperature of the polynucleotide synthesis reaction is increased to the optimal priming and or extension temperature of the processive polymerase to achieve highly processive specific amplification, the thermally sensitive bolcker protein becomes temperature inactivated and no longer binds primed substrate so the processive polymerase can extend or amplify the desired target.

Preferably, the blocking polymerase is more efficient at binding primed substrate (i.e., binds with greater affinity) at lower temperatures than the processive polymerase. Alternatively, if the blocking polymerase does not bind primed template with higher affinity than the thermostable processive polymerase, then higher amounts of the blocking polymerase can be used. In either case, it is preferred that the blocking polymerase can out compete the thermostable processive polymerase for primed substrate, making it unavailable to the processive polymerase until the blocking polymerase becomes inactivated and can no longer bind the primed substrate. Preferably, the blocking polymerase is 3′ to 5′ exonuclease free or has reduced 3′ to 5′ exonuclease activity, which can degrade primers, lower yield, and reduce the specificity of priming. However, deficient exonuclease activity is not required to achieve hot start using the compositions and methods of this invention. Hot start can still be achieved as long as the blocking polymerase can bind primed template. Ideally the blocking polymerase has optimal activity at 37° C. or lower, and is does not survive temperatures above 37° C. or routine PCR thermal cycling. The blocking polymerase of the invention acts by means of substrate (i.e., primed template) binding.

Exo (−) Klenow DNA polymerase is an example of a polymerase which has optimal activity at 37° C. and is more active at 25° C. than PfuTurbo® DNA polymerase and binds primed substrate more efficiently at 25° C. than PfuTurbo® DNA polymerase. Exo (−) Klenow DNA polymerase is also an example of a thermally sensitive polymerase (i.e., it doesn't survive PCR thermal cycling).

The thermally sensitive blocking polymerase is used to achieve hot start by blending it with the processive polymerase that will be used in a polynucleotide synthesis reaction, so that the two proteins are added to the reaction mixture in a single solution, or are added at approximately the same time. Alternatively, the blocking polymerase is added to the reaction mix separately from the processive polymerase. The blocking polymerase is preferably added during setup of the polynucleotide synthesis reaction, either before or simultaneously with the processive polymerase, in order to stop any primer extension during reaction setup.

Kits

The invention provides novel compositions and methods for hot start polynucleotide synthesis reactions such as primer extension reactions and PCR. The invention also contemplates a kit format which comprises a package unit having one or more containers of the subject composition and in some embodiments including containers of various reagents used for polynucleotide synthesis, including synthesis in PCR. In a preferred embodiment, the kit contains a blocking polymerase which is a mutant derived from the Klenow fragment of E. coli Pol I. In a more preferred embodiment the blocking polymerase is a polypeptide comprising the amino acid sequence shown in SEQ ID NO:1. In some versions of the kit, the blocking polymerase is a nucleic acid polymerase and the kit includes an antibody that partially or completely inhibits the polymerase activity of the blocking polymerase. The kit may also contain one or more of the following items: polymerization enzymes, polynucleotide precursors, primers, buffers, instructions, and controls. The kits may include containers of reagents mixed together in suitable proportions for performing the methods in accordance with the invention. Reagent containers preferably contain reagents in unit quantities that obviate measuring steps when performing the subject methods. One kit according to the invention also contains a DNA yield standard for the quantitation of the PCR product yields from a stained gel.

In one preferred embodiment, the kit contains a thermally sensitive blocking polymerase protein. In a more preferred embodiment, the blocking polymerase protein contained in the kit is the D705P (exo −) mutant of the Klenow fragment of E. coli DNA polymerase I. In another preferred embodiment, the kit comprises both a thermally sensitive blocking polymerase protein and a thermostable nucleic acid polymerase. In a more preferred embodiment, the blocking polymerase protein contained in the kit is the D705P (exo −) mutant of the Klenow fragment of E. coli DNA polymerase I and the polymerase is Taq polymerase, Pfu polymerase, or a mixture of Taq and Pfu polymerases.

### EXAMPLES

The examples below are non-limiting and are merely representative of various aspects and features of the subject invention.

### Example 1

Construction of a Functionally Deficient DNA Polymerase

To eliminate the polymerase activity from exo (−) Klenow DNA polymerase without affecting substrate binding, aspartic acid 705 in the α-helix in motif A in the palm subdomain, which controls dNTP interaction, was mutated to proline. The D705P exo(−) Klenow mutant was expressed and purified and tested for polymerase activity using the primed polynucleotide substrate M13 in the primed M13 DNA polymerase activity assay. The D705P exo(−) Klenow DNA polymerase demonstrated approximately 99.6% loss of DNA polymerase activity at 25° C. compared to the unmutated exo(−) Klenow DNA polymerase (FIG. 3A) and 95% loss of DNA polymerase activity compared with PfuTurbo® DNA polymerase at 25° C. (FIG. 3B).

### Example 2

Demonstration of Primed Substrate Blocking by D705P exo(−) Klenow DNA Polymerase

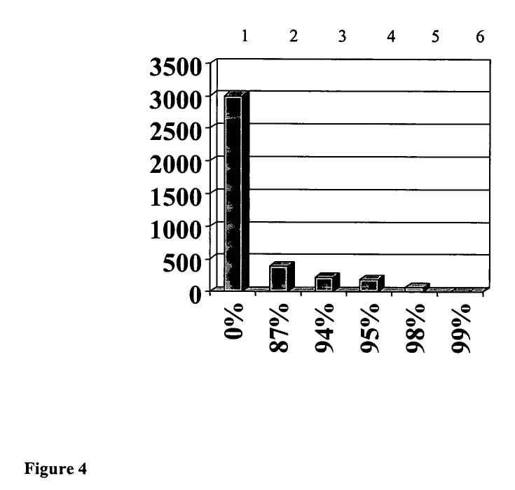

Substrate blocking was measured by loss of polymerase incorporated counts by PfuTurbo® DNA polymerase as was titrated into the reaction. The more D705P exo(−) Klenow was added, the less primed substrate was available to PfuTurbo®. D705P exo(−) Klenow DNA polymerase was added to primed M13 DNA polymerase reactions with PfuTurbo® DNA polymerase and incubated at 25° C. 1.25 units of PfuTurbo DNA polymerase was mixed with 0 ng, 7.5 ng, 15 ng, 30 ng, 60 ng, and 90 ng of D705P exo(−) Klenow DNA polymerase and incubated at 25° C. for 3 hours. The results are shown in FIG. 4. 0 ng D705P exo(−) Klenow positive control resulted in a 0% loss of polymerase incorporation by PfuTurbo. 7.5 ng of D705P exo(−) Klenow resulted in 87% loss of polymerase incorporation by PfuTurbo. 15 ng of D705P exo(−) Klenow resulted in 94% loss of polymerase incorporation by PfuTurbo. 30 ng of D705P exo(−) Klenow resulted in 95% loss of polymerase incorporation by PfuTurbo. 60 ng of D705P exo(−) Klenow resulted in 98% loss of polymerase incorporation by PfuTurbo. 90 ng of D705P exo(−) Klenow resulted in 99% loss of polymerase incorporation by PfuTurbo DNA polymerase.

### Example 3

Demonstration of Hot Start PCR Amplification with The D705P exo(−) Klenow Mutant

PCR hot start was demonstrated using an HIV gag gene hot start amplification system and PfuTurbo® DNA polymerase. In this PCR assay a limiting amount (50 copies) of specific HIV gag gene template was amplified from a background of nonspecific denatured human genomic DNA template. The primer sequences were as follows: Forward, 5′-ATAATCCACCTATCCCAGTAGGAGAAAT-3′ (SEQ ID NO:3) and Reverse, 5′-TTTGGTCCTTGTCTTATGTCCAGAATGC-3′ (SEQ ID NO:4). The gag specific PCR primers readily and nonspecifically primed the denatured human DNA template at 25° C., generating copious amounts of primed substrate that PfuTurbo DNA polymerase extends. These nonspecific polynucleotide extension products were then amplified into nonspecific PCR products during PCR amplification and inhibited the specific amplification of the desired HIV gag gene. In the presence of the hot start blocking polymerase protein, the non-specifically primed substrates were not extended; therefore few or no non-specific amplification products were generated and amplification of the HIV gag gene was achieved.

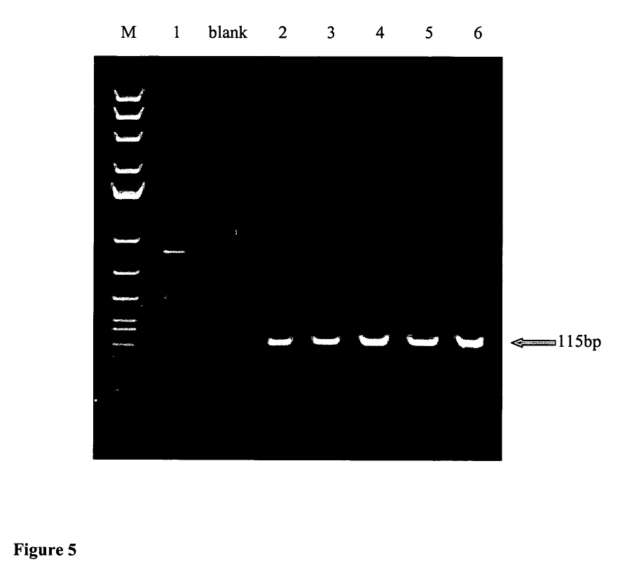

2.5 units of PfuTurbo® was mixed with 0 ng, 7.5 ng, 15 ng, and 30 ng of the D705P exo(−) Klenow mutant DNA polymerase in PCR reactions. PfuTurbo® Hot Start DNA polymerase (PfuTurbo® DNA polymerase with anti-Pfu hot start antibody) was included as a positive hot start control. The PCR reactions were incubated at 25° C. for 15 minutes to allow nonspecific primer annealing and extension. The PCR reactions were then placed in a thermal cycler and cycled using optimal conditions for the specific amplification of HIV gag. One cycle was performed at 95° C. for 2 minutes (initial denaturation of template DNA and deactivation of blocking polymerase), followed by 35 cycles at 95° C. for 30 seconds (denaturation), 58° C. for 30 seconds (annealing), 72° C. for 1 minute (extension), followed by 1 cycle at 72° C. for 5 minutes (final extension). The results are shown in FIG. 5. The 0 ng D705P exo(−) Klenow mutant non-hot start control reaction generated numerous nonspecific bands and the HIV gag gene failed to amplify. The reactions with 7.5-60 ng of the D705P exo(−) Klenow mutant all demonstrated hot start activity and the HIV gag gene was successfully amplified in all reactions. The PCR reactions with 30 ng and 60 ng of the D705P exo(−) Klenow mutant demonstrated equal hot start capability compared to the hot start antibody technology.

### Example 4

Comparison of the D705P exo(−) Klenow Mutant Hot Start to Antibody Hot Start for Universal Hot Start Capability

Universal hot start capability was evaluated with a proofreading DNA polymerase, a non-proofreading DNA polymerase and a DNA polymerase blend using the HIV gag hot start PCR assay. PfuTurbo® DNA polymerase represented the proofreader, Taq 2000® DNA polymerase represented the non-proofreader, and Herculase® DNA polymerase represented the DNA polymerase blend. D705P exo(−) Klenow mutant hot start and antibody hot start were compared for all DNA polymerases.

The proofreading DNA polymerase hot start was evaluated by amplifying the HIV gag target with 2.5 units of PfuTurbo® mixed with 0 ng and 30 ng of D705P exo(−) Klenow mutant DNA polymerase and compared to the amplification with 2.5 units of PfuTurbo® Hot Start DNA polymerase with hot start antibody. The results are seen in FIG. 5. The 0 ng D705P exo(−) Klenow mutant negative hot start control reaction demonstrated no hot start activity and generated multiple non specific bands. The 30 ng D705P exo(−) Klenow mutant reaction and the hot Start antibody reaction both demonstrated equivalent hot start activity and amplified the specific HIV gag target with equal specificity and yields.

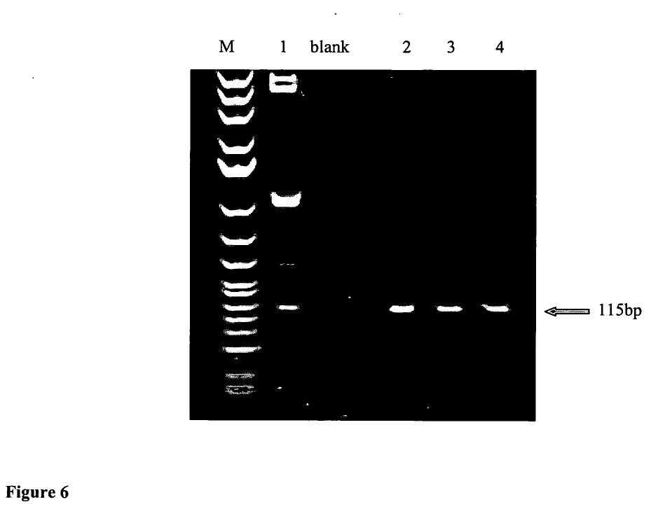

The non-proofreading DNA polymerase hot start was evaluated by amplifying the HIV gag target with 2.5 units of Taq 2000® mixed with 0 ng, 3.5 ng, and 7.5 ng of D705P exo(−) Klenow mutant DNA polymerase and compared to amplification with 2.5 units of Taq 2000® DNA polymerase pre-incubated with 100 ng of anti Taq hot start antibody. The results are shown in FIG. 6. 100 ng of hot start antibody completely inactivated 2.5 units of Taq 2000® and provided complete hot start. The 0 ng D705P exo(−) Klenow mutant negative hot start control reaction demonstrated no hot start activity and generated multiple non specific bands. The reactions with 3.5 ng and 7.5 ng of the D705P exo(−) Klenow mutant and the Taq 2000® antibody hot start reaction all demonstrated equivalent hot start activity and amplified the specific HIV gag target with equal specificity and yields (FIG. 6).

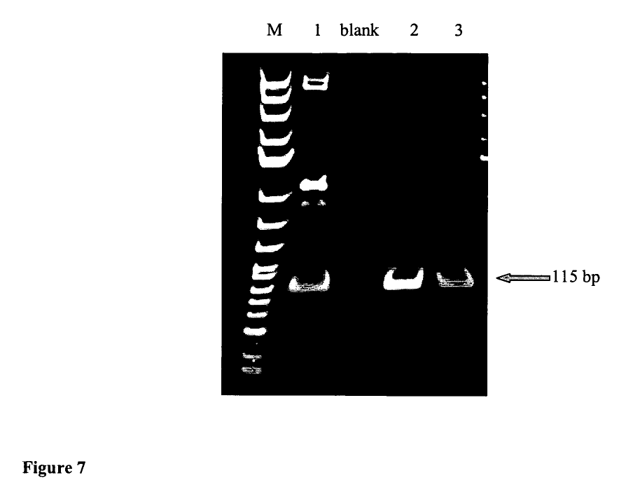

The DNA polymerase blend hot start was evaluated by amplifying the HIV gag target with 2.5 units of Herculase® DNA polymerase mixed with 0 ng and 60 ng of D705P exo(−) Klenow mutant DNA polymerase. This was compared to the amplification of HIV gag with 2.5 units of Herculase® Hot Start DNA polymerase with hot start antibody. The results are shown in FIG. 7. The 0 ng D705P exo(−) Klenow mutant negative hot start control reaction demonstrated no hot start activity and generated multiple non specific bands. The 60 ng D705P exo(−) Klenow mutant reaction and the Herculase® hot start antibody reaction both demonstrated equivalent hot start activity and amplified the specific HIV gag target with equal specificity and yields.

1. A nucleic acid molecule comprising a nucleotide sequence that encodes the amino acid sequence shown in SEQ ID NO:1.

2. The nucleic acid molecule of claim 1 comprising the nucleotide sequence shown in SEQ ID NO:2.

3. A vector comprising the nucleic acid molecule of claim 1.

4. A host cell comprising the vector of claim 3.

5. A method of making a polypeptide comprising the amino acid sequence shown in SEQ ID NO:1, the method comprising culturing the host cell of claim 4 under conditions suitable for the expression of said polypeptide.

6. A polypeptide comprising the amino acid sequence shown in SEQ ID NO:1.

7. A blocking polymerase which binds to a primed polynucleotide template and blocks elongation of the primer by a thermostable polymerase, wherein the blocking polymerase has an inactivation temperature above 37 C.

8. The blocking polymerase of claim 7 which has an inactivation temperature above 45° C.

9. The blocking polymerase of claim 8 which has an inactivation temperature above 50° C.

10. The blocking polymerase of claim 9 which has an inactivation temperature above 60° C.

11. The blocking polymerase of claim 10 which has an inactivation temperature above 70° C.

12. The blocking polymerase of claim 11 which has an inactivation temperature above 80° C.

13. A composition comprising the blocking polymerase of claim 7 and a thermostable processive DNA polymerase.

14. The composition of claim 13, wherein the priming temperature of the processive polymerase is higher than the inactivation temperature of the blocking polymerase.

15. The composition of claim 13, wherein the concentration of the blocking polymerase is greater than the concentration of the processive polymerase.

16. The composition of claim 13, wherein the concentration of the blocking polymerase is less than or equal to the concentration of the processive polymerase, and wherein the blocking polymerase has a higher affinity than the processive polymerase for binding to said primed polynucleotide template at temperatures below the inactivation temperature of the blocking polymerase.

17. The composition of claim 13, wherein the blocking polymerase has a higher affinity or the same affinity compared with the processive polymerase for binding to said primed polynucleotide template at temperatures below the inactivation temperature of the blocking polymerase.

18. The blocking polymerase of claim 7 which is a functionally deficient nucleic acid polymerase.

19. The blocking polymerase of claim 18 which is non-processive.

20. The blocking polymerase of claim 7 which is an isolated naturally occuring polypeptide.

21. The blocking polymerase of claim 18 which is devoid of 3′ to 5′ exonuclease activity.

22. A functionally deficient mutant DNA polymerase which, compared with the DNA polymerase from which it was derived, has reduced polymerase activity and reduced processivity; wherein said mutant DNA polymerase is capable of binding a primed polynucleotide template; and wherein said mutant DNA polymerase has an inactivation temperature above 37° C.

23. The mutant polymerase of claim 22 which has no polymerase activity.

24. The mutant polymerase of claim 22 which has reduced 3′ to 5′ exonuclease activity compared with the DNA polymerase from which it was derived.

25. The mutant polymerase of claim 24 which has no 3′ to 5′ exonuclease activity.

26. The mutant polymerase of claim 22 which is non-processive.

27. The mutant polymerase of claim 24 which is derived from the Klenow fragment of E. coli DNA pol I.

28. The mutant polymerase of claim 22 which has an inactivation temperature above 45° C.

29. The mutant polymerase of claim 28 which has an inactivation temperature above 50° C.

30. The mutant polymerase of claim 29 which has an inactivation temperature above 60° C.

31. The mutant polymerase of claim 30 which has an inactivation temperature above 70° C.

32. The mutant polymerase of claim 31 which has an inactivation temperature above 80° C.

33. A composition comprising the mutant polymerase of claim 22 and a thermostable processive DNA polymerase.

34. The composition of claim 33, wherein the priming temperature of the processive polymerase is higher than the inactivation temperature of the mutant polymerase.

35. The composition of claim 33, wherein the concentration of mutant polymerase is greater than the concentration of the processive polymerase.

36. The composition of claim 33, wherein the concentration of mutant polymerase is less than or equal to the concentration of the processive polymerase, and wherein the mutant polymerase has a higher affinity than the processive polymerase for binding to said primed polynucleotide template at temperatures below the inactivation temperature of the mutant polymerase.

37. The composition of claim 33, wherein the mutant polymerase has a higher affinity or the same affinity compared with the processive polymerase for binding to a primed polynucleotide template at temperatures below the inactivation temperature of the mutant polymerase.

38. The composition of claim 33, wherein the thermostable processive polymerase is selected from the group consisting of Taq DNA polymerase, Pfu DNA polymerase, and a mixture of Taq and Pfu DNA polymerases.

39. The composition of claim 33, wherein the mutant polymerase is derived from the Klenow fragment of E. coli DNA pol I.

40. A method of primer extension comprising extending an oligonucleotide primer which is annealed to a nucleic acid template using a mixture of a thermostable processive polymerase and the blocking polymerase of claim 7, wherein the blocking polymerase is added prior to initiating the extension reaction, wherein the reaction mixture is below the inactivation temperature of the blocking polymerase prior to initiating the extension reaction, and wherein the extension reaction is performed at a temperature above the inactivation temperature of the blocking polymerase.

41. The method of claim 40, wherein the thermostable processive polymerase and the blocking polymerase are added to the reaction mixture at about the same time.

42. The method of claim 40, wherein the blocking polymerase is added to the reaction mixture prior to adding the thermostable processive polymerase.

43. The method of claim 40, wherein the blocking polymerase is a nucleic acid polymerase, and wherein the reaction mixture further comprises an antibody which inhibits the polymerase activity of the blocking polymerase.

44. The method of claim 40, wherein the inactivation temperature of the blocking polymerase is at least 50° C.

45. The method of claim 44, wherein the inactivation temperature of the blocking polymerase is at least 60° C.

46. The method of claim 45, wherein the inactivation temperature of the blocking polymerase is at least 70° C.

47. The method of claim 46, wherein the inactivation temperature of the blocking polymerase is at least 80° C.

48. The method of claim 40, wherein the blocking polymerase is a functionally deficient mutant DNA polymerase.

49. The method of claim 48, wherein the mutant DNA polymerase comprises the amino acid sequence shown in SEQ ID NO:1.

50. The method of claim 40, wherein the thermostable processive polymerase is selected from the group consisting of Taq DNA polymerase, Pfu DNA polymerase, and a mixture of Taq and Pfu DNA polymerases.

51. A method of hot start polymerase chain reaction (PCR) comprising performing a PCR reaction in the presence of a thermostable processive polymerase and the blocking polymerase of claim 7, wherein the blocking polymerase is added prior to initiating the first extension reaction, wherein the reaction mixture is below the inactivation temperature of the blocking polymerase prior to initiating the first extension reaction, and wherein the first extension reaction is performed at a temperature above the inactivation temperature of the blocking polymerase.

52. The method of claim 51, wherein the thermostable processive polymerase and the blocking polymerase are added to the reaction mixture at about the same time.

53. The method of claim 51, wherein the blocking polymerase is added to the reaction mixture prior to adding the thermostable processive polymerase.

54. The method of claim 51, wherein the blocking polymerase is a nucleic acid polymerase and the reaction mixture further comprises an antibody which inhibits the polymerase activity of the blocking polymerase.

55. The method of claim 51, wherein the inactivation temperature of the blocking polymerase is at least 50° C.

56. The method of claim 55, wherein the inactivation temperature of the blocking polymerase is at least 60° C.

57. The method of claim 56, wherein the inactivation temperature of the blocking polymerase is at least 70° C.

58. The method of claim 57, wherein the inactivation temperature of the blocking polymerase is at least 80° C.

59. The method of claim 51, wherein the blocking polymerase is a functionally deficient mutant DNA polymerase.

60. The method of claim 59, wherein the mutant polymerase is derived from the Klenow fragment of E. coli DNA pol I.

61. The method of claim 59, wherein the mutant DNA polymerase comprises the amino acid sequence shown in SEQ ID NO:1.

62. The method of claim 59, wherein a PCR enhancing factor is added to the reaction mixture.

63. A kit comprising a blocking polymerase and packaging therefor; wherein the blocking polymerase binds to a primed polynucleotide template and blocks elongation of the primer by a thermostable polymerase, and wherein the blocking polymerase has an inactivation temperature above 37 C.

64. The kit of claim 63 further comprising a thermostable processive polymerase.

65. The kit of claim 63 further comprising a PCR enhancing factor.

66. The kit of claim 63, wherein the blocking polymerase has an inactivation temperature above 45° C.

67. The kit of claim 66, wherein the blocking polymerase has an inactivation temperature above 50° C.

68. The kit of claim 67, wherein the blocking polymerase has an inactivation temperature above 60° C.

69. The kit of claim 68, wherein the blocking polymerase has an inactivation temperature above 70° C.

70. The kit of claim 69, wherein the blocking polymerase has an inactivation temperature above 80° C.

71. The kit of claim 64, wherein the thermostable processive polymerase and the blocking polymerase are provided in the same solution.

72. The kit of claim 71, wherein the concentration of the blocking polymerase is greater than the concentration of the thermostable processive polymerase.

73. The kit of claim 64, wherein the thermostable processive polymerase and the blocking polymerase are provided in separate solutions.

74. The kit of claim 63, wherein the blocking polymerase is a functionally deficient nucleic acid polymerase.

75. The kit of claim 74, wherein the functionally deficient nucleic acid polymerase is a mutant derived from the Klenow fragment of E. coli DNA pol I.

76. The kit of claim 74, wherein the blocking polymerase is non-processive.

77. The kit of claim 74, wherein the blocking polymerase is devoid of 3′ to 5′ exonuclease activity.

78. The kit of claim 74, wherein the blocking polymerase is a polypeptide comprising the amino acid sequence shown in SEQ ID NO:1.

79. The kit of claim 64, wherein the priming temperature of the thermostable processive polymerase is higher than the inactivation temperature of the blocking polymerase.

80. The kit of claim 64, wherein the blocking polymerase has a higher affinity than the thermostable processive polymerase for binding to said primed polynucleotide template at temperatures below the inactivation temperature of the blocking polymerase.

81. The kit of claim 64, wherein the thermostable processive polymerase is selected from the group consisting of Taq DNA polymerase, Pfu DNA polymerase, and a mixture of Taq and Pfu DNA polymerases.

82. The kit of claim 63, further comprising a PCR buffer and a mixture of deoxyribonucleotides.

83. The kit of claim 63, further comprising a pair of primers designed to amplify a selected target nucleic acid sequence using PCR.


SEQUENCE LISTING (4 sequences)

SEQ ID NO: 1 - protein, 928 aa, Artificial
  Derived from the Klenow fragment of E. coli Pol I
MVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSAPYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 2 - DNA, 2787 bp, Artificial
  Coding sequence for SEQ ID NO1
atggttcagatcccccaaaatccacttatccttgtagatggttcatcttatctttatcgcgcatatcacgcgtttcccccgctgactaacagcgcaggcgagccgaccggtgcgatgtatggtgtcctcaacatgctgcgcagtctgatcatgcaatataaaccgacgcatgcagcggtggtctttgacgccaagggaaaaacctttcgtgatgaactgtttgaacattacaaatcacatcgcccgccaatgccggacgatctgcgtgcacaaatcgaacccttgcacgcgatggttaaagcgatgggactgccgctgctggcggtttctggcgtagaagcggacgacgttatcggtactctggcgcgcgaagccgaaaaagccgggcgtccggtgctgatcagcactggcgataaagatatggcgcagctggtgacgccaaatattacgcttatcaataccatgacgaataccatcctcggaccggaagaggtggtgaataagtacggcgtgccgccagaactgatcatcgatttcctggcgctgatgggtgactcctctgataacattcctggcgtaccgggcgtcggtgaaaaaaccgcgcaggcattgctgcaaggtcttggcggactggatacgctgtatgccgagccagaaaaaattgctgggttgagcttccgtggcgcgaaaacaatggcagcgaagctcgagcaaaacaaagaagttgcttatctctcataccagctggcgacgattaaaaccgacgttgaactggagctgacctgtgaacaactggaagtgcagcaaccggcagcggaagagttgttggggctgttcaaaaagtatgagttcaaacgctggactgctgatgtcgaagcgggcaaatggttacaggccaaaggggcaaaaccagccgcgaagccacaggaaaccagtgttgcagacgaagcaccagaagtgacggcaacggtgatttcttatgacaactacgtcaccatccttgatgaagaaacactgaaagcgtggattgcgaagctggaaaaagcgccggtatttgcatttgctaccgcaaccgacagccttgataacatctctgctaacctggtcgggctttcttttgctatcgagccaggcgtagcggcatatattccggttgctcatgattatcttgatgcgcccgatcaaatctctcgcgagcgtgcactcgagttgctaaaaccgctgctggaagatgaaaaggcgctgaaggtcgggcaaaacctgaaatacgatcgcggtattctggcgaactacggcattgaactgcgtgggattgcgtttgataccatgctggagtcctacattctcaatagcgttgccgggcgtcacgatatggacagcctcgcggaacgttggttgaagcacaaaaccatcacttttgaagagattgctggtaaaggcaaaaatcaactgacctttaaccagattgccctcgaagaagccggacgttacgccgccgaagatgcagatgtcaccttgcagttgcatctgaaaatgtggccggatctgcaaaaacacaaagggccgttgaacgtcttcgagaatatcgaaatgccgctggtgccggtgctttcacgcattgaacgtaacggtgtgaagatcgatccgaaagtgctgcacaatcattctgaagagctcacccttcgtctggctgagctggaaaagaaagcgcatgaaattgcaggtgaggaatttaacctttcttccaccaagcagttacaaaccattctctttgaaaaacagggcattaaaccgctgaagaaaacgccgggtggcgcgccgtcaacgtcggaagaggtactggaagaactggcgctggactatccgttgccaaaagtgattctggagtatcgtggtctggcgaagctgaaatcgacctacaccgacaagctgccgctgatgatcaacccgaaaaccgggcgtgtgcatacctcttatcaccaggcagtaactgcaacgggacgtttatcgtcaaccgatcctaacctgcaaaacattccggtgcgtaacgaagaaggtcgtcgtatccgccaggcgtttattgcgccagaggattatgtgattgtctcagcgccctactcgcagattgaactgcgcattatggcgcatctttcgcgtgacaaaggcttgctgaccgcattcgcggaaggaaaagatatccaccgggcaacggcggcagaagtgtttggtttgccactggaaaccgtcaccagcgagcaacgccgtagcgcgaaagcgatcaactttggtctgatttatggcatgagtgctttcggtctggcgcggcaattgaacattccacgtaaagaagcgcagaagtacatggacctttacttcgaacgctaccctggcgtgctggagtatatggaacgcacccgtgctcaggcgaaagagcagggctacgttgaaacgctggacggacgccgtctgtatctgccggatatcaaatccagcaatggtgctcgtcgtgcagcggctgaacgtgcagccattaacgcgccaatgcagggaaccgccgccgacattatcaaacgggcgatgattgccgttgatgcgtggttacaggctgagcaaccgcgtgtacgtatgatcatgcaggtacacgatgaactggtatttgaagttcataaagatgatgttgatgccgtcgcgaagcagattcatcaactgatggaaaactgtacccgtctggatgtgccgttgctggtggaagtggggagtggcgaaaactgggatcaggcgcactaa

SEQ ID NO: 3 - DNA, 28 bp, Artificial
  PCR primer
ataatccacctatcccagtaggagaaat

SEQ ID NO: 4 - DNA, 28 bp, Artificial
  PCR primer
tttggtccttgtcttatgtccagaatgc